(19) United States
(12) Patent Application Publication
(10) Pub. No.: US 2021/0059762 A1
(43) Pub. Date: Mar. 4, 2021

(54) MOTION COMPENSATION PLATFORM FOR IMAGE GUIDED PERCUTANEOUS ACCESS TO BODILY ORGANS AND STRUCTURES

(71) Applicants: Changi General Hospital Pte Ltd, Singapore (SG); Singapore University of Technology and Design, Singapore (SG)

(72) Inventors: Foo Cheong NG, Singapore (SG); Sey Kiat Terence LIM, Singapore (SG); Subburaj KARUPPPASAMY, Singapore (SG); U-Xuan TAN, Singapore (SG); Lujie CHEN, Singapore (SG); Shaohui FOONG, Singapore (SG); Liangjing YANG, Singapore (SG); Hsieh-Yu LI, Singapore (SG); Ishara Chaminda Kariyawasam PARANAWITHANA, Singapore (SG); Zhong Hoo CHAU, Singapore (SG); Muthu Rama Krishnan MOOKIAH, Singapore (SG)

(21) Appl. No.: 16/958,587

(22) PCT Filed: Dec. 28, 2018
(86) PCT No.: PCT/SG2018/050637

§ 371 Date: Jun. 26, 2020

(30) Foreign Application Priority Data
Dec. 28, 2017 (SG) 10201710888P

(51) Int. Cl. A61B 34/20; A61B 5/00; A61B 5/055; A61B 6/03; A61B 8/08; A61B 34/00; A61B 17/34; A61B 34/10; A61B 90/00; G06T 7/33; G06T 3/40; G06T 7/11; G06T 17/20

(52) U.S. Cl. CPC A61B 34/20; A61B 34/30; A61B 5/055; A61B 6/032; A61B 8/52; A61B 34/70; A61B 17/3403; A61B 34/10; A61B 90/37; A61B 34/25; A61B 5/7425; G06T 7/344; G06T 3/40; G06T 7/11; G06T 17/20; A61B 34/76; A61B 5/0037

(57) ABSTRACT

A method and a system for registering real-time intra-operative image data of a body to a model of the body, the method comprising, segmenting a plurality of image data of the body obtained using a pre-operative imaging device; constructing the model of the body from the segmented plurality of image data; identifying one or more landmark features on the model of the body; acquiring the real-time intra-operative image data of the body using an intra-operative imaging device; and registering the real-time intra-operative image data of the body to the model of the body by matching one or more landmark features labelled on the real-time intra-operative image data to one or more corresponding landmark features on the model of the body, wherein the one or more landmark features comprises a superior and an inferior pole of the body.

### TECHNICAL FIELD

The present disclosure relates broadly to a method and system for registering real-time intra-operative image data of a body to a model of the body, as well as an apparatus for tracking a target in a body behind a surface using an intra-operative imaging device.

### BACKGROUND

Image-guided surgery has expanded significantly into a number of clinical procedures due to significant advances in computing power, high-resolution medical imaging modalities, and scientific visualisation methods. In general, the main components of an image-guided surgical system comprise identifying anatomical bodies/regions of interest to excise or focus, preoperative modelling e.g. three-dimensional (3D) modelling of anatomical models and virtual surgery planning, intra-operative registration of pre-planned surgical procedure and 3D models with continuous images, and performing the surgical procedure in accordance with the pre-planning.

Intra-operative registration is considered an important process in any image-guided/computer aided surgical process. This is because the accuracy of the registration process directly correlates with the precision of mapping of a pre-planned surgical procedure, visualization of lesions or regions of interest, and guidance with respect to a subject or patient. However, intra-operative image registration faces challenges such as an excessive need for manual intervention, extensive set-up time and amount of effort required.

Historically, fluoroscopy imaging modality has been used as real-time/live imaging for registering pre-operative plans to guide through the procedure. However, there are problems to this approach such as the initial investment and operating costs, the use of expensive and bulky equipment, and exposure of the patient and surgical staff to unnecessary ionising radiation during the procedure.

Several methods have been proposed and developed for intra-operative registration of preoperative image volumes with fiducial-based registration (i.e. physical markers are placed on the patient, either during or before the surgical procedure). Fiducial points are marked and labelled in the pre-operative images or reconstructed 3D anatomical models from those images. During the surgical procedure, the same anatomical landmarks or fiducial points are localized and labelled on the patient for reference. Typically, only a few anatomical landmarks can be reliably selected due to anatomical variations. Therefore, most of the proposed methods have focused on the use of artificial fiducial markers on the external surface of the patient instead of intra-operative labelling after opening up the patient. While intra-operative labelling after opening up the patient may be an accurate registration approach, it increases the complexity of the surgical procedure and the risks of complications due to the level of invasiveness required to reach each fiducial point directly on the patient.

Thus, there is a need for a method and system for registering real-time intra-operative image data of a body to a model of the body, as well as an apparatus for tracking a target in a body behind a surface using an intra-operative imaging device.

### SUMMARY

According to one aspect, there is provided a method for registering real-time intra-operative image data of a body to a model of the body, the method comprising, segmenting a plurality of image data of the body obtained using a pre-operative imaging device; constructing the model of the body from the segmented plurality of image data; identifying one or more landmark features on the model of the body; acquiring the real-time intra-operative image data of the body using an intra-operative imaging device; and registering the real-time intra-operative image data of the body to the model of the body by matching one or more landmark features labelled on the real-time intra-operative image data to one or more corresponding landmark features on the model of the body, wherein the one or more landmark features comprises a superior and an inferior pole of the body.

The one or more landmark features may further comprise a line connecting the superior and inferior poles of the body.

The one or more landmark features may further comprise a combination of saddle ridge, saddle valley, peak and/or pit.

The step of identifying one or more landmark features may comprise calculating one or more principal curvatures for each vertex of the body.

The step of identifying one or more landmark features may further comprise calculating the Gaussian and mean curvatures using the one or more principal curvatures, wherein the one or more landmark features is identified by a change in sign of the Gaussian and mean curvatures.

The method may further comprise labelling one or more landmark features on the real-time intra-operative image data using a user interface input module.

The method may further comprise sub-sampling or down-sampling of the model to match the resolution of the real-time intra-operative image data acquired by the intra-operative imaging device.

The step of registering may comprise iteratively reducing the Euclidean distance between the one or more landmark features labelled on the real-time intra-operative image data of the body and the one or more corresponding landmark features on the model of the body.

The step of registering may comprise matching the superior and inferior poles of the body on the real-time intra-operative image data to the respective superior and inferior poles of the body on the model of the body.

The step of segmenting may comprise introducing one or more seed points in one or more regions of interest, wherein each of the one or more seed points comprises a pre-defined threshold range of pixel intensities.

The method may further comprise iteratively adding to the one or more seed points, neighbouring voxels with pixel intensities within the pre-defined threshold range of pixel intensities of the one or more seed points.

The method may further comprise generating a polygonal mesh of the model to render the model for visualization on a display screen, wherein the polygonal mesh is a triangular or quadrilateral mesh.

The pre-operative imaging device may be a computed tomography (CT) imaging device, a magnetic resonance (MR) imaging device, or an ultrasound imaging device.

The intra-operative imaging device may be an ultrasound imaging device.

The body may be located within a human or an animal.

The method may further comprise labelling the one or more landmark features on the real-time intra-operative image data at substantially the same point in a respiratory cycle of the human or animal body.

The point in the respiratory cycle of the human or animal body may be the point of substantially maximum exhalation.

The body may be a kidney.

According to another aspect, there is provided a system for registering real-time intra-operative image data of a body to a model of the body, the system comprising, an image processing module configured to: segment a plurality of image data of the body obtained using a pre-operative imaging device; construct the model of the body from the segmented plurality of image data; identify one or more landmark features on the model of the body; an intra-operative imaging device configured to acquire the real-time intra-operative image data of the body; and a registration module configured to register the real-time intra-operative image data of the body to the model of the body by matching one or more landmark features labelled on the real-time intra-operative image data to one or more corresponding landmark features on the model of the body, wherein the one or more landmark features comprises a superior and an inferior pole of the body.

The one or more landmark features may further comprise a line connecting the superior and inferior poles of the body.

The one or more landmark features may further comprise a combination of saddle ridge, saddle valley, peak and/or pit.

The image processing module may be configured to calculate one or more principal curvatures for each vertex of the body.

The image processing module may be further configured to calculate the Gaussian and mean curvatures using the one or more principal curvatures, wherein the one or more landmark features is identified by a change in sign of the Gaussian and mean curvatures.

The system may further comprise a user interface input module configured to facilitate labelling of one or more landmark features on the real-time intra-operative image data.

The image processing module may be configured to perform sub-sampling or down-sampling of the model to match the resolution of the real-time intra-operative image data acquired by the intra-operative imaging device.

The registration module may be configured to iteratively reduce the Euclidean distance between the one or more landmark features labelled on the real-time intra-operative image data of the body and the one or more corresponding landmark features on the model of the body.

The registration module may be configured to match the superior and inferior poles of the body on the real-time intra-operative image data to the respective superior and inferior poles of the body on the model of the body.

The image processing module may be configured to introduce one or more seed points in one or more regions of interest, wherein each of the one or more seed points comprises a pre-defined threshold range of pixel intensities.

The image processing module may be further configured to iteratively add to the one or more seed points, neighbouring voxels with pixel intensities within the pre-defined threshold range of pixel intensities of the one or more seed points.

The image processing module may be further configured to generate a polygonal mesh of the model to render the model for visualization on a display screen, wherein the polygonal mesh is a triangular or quadrilateral mesh.

The system may further comprise a pre-operative image device for acquiring a plurality of image data of the body, wherein the pre-operative imaging device is a computed tomography (CT) imaging device, a magnetic resonance (MR) imaging device, or an ultrasound imaging device.

The intra-operative imaging device may be an ultrasound imaging device.

The body may be located within a human or an animal.

The one or more landmark features may be labelled on the real-time intra-operative image data at substantially the same point in a respiratory cycle of the human or animal body.

The point in the respiratory cycle of the human or animal body may be the point of substantially maximum exhalation.

The body may be a kidney.

According to another aspect, there is provided an apparatus for tracking a target in a body behind a surface using an intra-operative imaging device, the intra-operative imaging device comprising a probe for performing scans of the body, and an image feedback unit for providing real-time intra-operative image data of the scans obtained by the probe, the apparatus comprising, a manipulator for engaging and manipulating the probe; a control unit for positioning the probe by controlling the manipulator, said control unit comprising, an image processing module configured to: segment a plurality of image data of the body obtained using a pre-operative imaging device; construct a model of the body from the segmented plurality of image data, said model comprising an optimal needle trajectory information, and said optimal needle trajectory information comprising positional information on a point on the surface and a point of the target; identify one or more landmark features on the model of the body; a registration module configured to register the real-time intra-operative image data of the body to the model of the body by matching one or more landmark features labelled on the real-time intra-operative image data to one or more corresponding landmark features on the model of the body, wherein the one or more landmark features comprises a superior and an inferior pole of the body; and a needle insert device coupled to the manipulator, said needle insert device comprising holding means for holding a needle at an angle directed at the target; wherein said manipulator is configured to directly manipulate the probe in collaboration with the control unit such that the needle substantially follows the optimal needle trajectory information to access the target in the body.

The control unit may comprise a collaborative controller for addressing undesired motion of the probe.

The collaborative controller may address undesired motion of the probe caused by the user or the body of the target.

The collaborative controller may regulate a force applied by the user on the manipulator.

The collaborative controller may further comprise a rotational motion control unit for regulating an angular velocity of rotational motions caused by the user manipulation; and a translational motion control unit for regulating the translational velocity of the translational motions caused by the user manipulation.

The control unit may further comprise an admittance controller for maintaining a desired force applied by the probe against the surface.

The admittance controller may comprise a force sensor for estimating environmental forces; a low pass filter for filtering the estimated environmental forces; and said admittance controller configured for providing the desired force against the contact surface, based on the filtered environmental forces.

The needle insertion device may further comprise driving means for driving a needle at the target, said needle held within the holding means.

The holding means may comprise a pair of friction rollers arranged in a side-by-side configuration with the respective rotational axis of the friction rollers in parallel, such that the needle can be held between the frictions rollers in a manner where the longitudinal axis of the needle is parallel with the rotational axis of the friction rollers; wherein each friction roller is rotatable about their respective axis such that rotation of the friction rollers in opposite directions moves the needle along its longitudinal axis.

The driving means may comprise a DC motor for rotating the friction rollers.

The holding means may further comprise an additional friction roller for assisting in needle alignment.

The holding means may further comprise biasing means to bias the needle between each of the friction rollers.

The DC motor may be controllable by a microprocessor, said microprocessor configured for controlling the rotation speed of the friction rollers, duration of movement, and direction of motor rotation.

The needle insertion device may comprise a mounting slot arranged for allowing the needle to be inserted such that the longitudinal axis of the needle is substantially perpendicular to the axis of the pair of friction rollers, by moving the needle in a direction perpendicular to the longitudinal axis of the needle.

According to another aspect, there is provided a non-transitory computer readable storage medium having stored thereon instructions for instructing a processing unit of a system to execute a method of registering real-time intra-operative image data of a body to a model of the body, the method comprising, segmenting a plurality of image data of the body obtained using a pre-operative imaging device; constructing the model of the body from the segmented plurality of image data; identifying one or more landmark features on the model of the body; acquiring the real-time intra-operative image data of the body using an intra-operative imaging device; and registering the real-time intra-operative image data of the body to the model of the body by matching one or more landmark features labelled on the real-time intra-operative image data to one or more corresponding landmark features on the model of the body, wherein the one or more landmark features comprises a superior and an inferior pole of the body.

### BRIEF DESCRIPTION OF THE DRAWINGS

Exemplary embodiments of the invention will be better understood and readily apparent to one of ordinary skill in the art from the following written description, by way of example only, and in conjunction with the drawings, in which:

FIG. 1 is a schematic flowchart for illustrating a process for registering real-time intra-operative image data of a body to a model of the body in an exemplary embodiment.

FIG. 2 is a screenshot of a graphical user interface (GUI) of a customised tool for performing interactive segmentation of a plurality of image data in an exemplary embodiment.

FIG. 3A is a processed CT image of a subject with a first segmentation view in an exemplary embodiment.

FIG. 3B is the processed CT image of the subject with a second segmentation view in the exemplary embodiment.

FIG. 4 is a 3D model of a kidney in an exemplary embodiment.

FIG. 5 is a set of images showing different curvature types by sign, in Gaussian and mean curvatures.

FIG. 6 is an ultrasound image labelled with a plurality of landmarks in an exemplary embodiment.

FIG. 7 is a composite image showing a 2D ultrasound image and 3D reconstructed model of a kidney after affine 3D-2D registration in an exemplary embodiment.

FIG. 8 is a schematic diagram of an overview of a system for implementing a method for tracking a target in a body behind a surface using an intra-operative imaging device in an exemplary embodiment.

FIG. 9A is a perspective view drawing of a robot for tracking a target in a body behind a surface using an intra-operative imaging device in an exemplary embodiment.

FIG. 9B is an enlarged perspective view drawing of an end effector of the robot in the exemplary embodiment.

FIG. 10 is a schematic diagram of a control scheme for rotational joints of a manipulator in a robot in an exemplary embodiment.

FIG. 11 is a schematic diagram of a control scheme for translational joints of a manipulator in a robot in an exemplary embodiment.

FIG. 12 is a graph of interactive force, Fᵢₙₜ against desired force, F_{des} and showing regions of dead zone, positive saturation and negative saturation in an exemplary embodiment.

FIG. 13 is a graph of system identification for one single axis—swept sine velocity experimental data obtained from an exemplary embodiment implementing the designed controllers, in comparison with the simulated data.

FIG. 14 is a graph showing stability and back-drivable analysis in an exemplary embodiment.

FIG. 15 is a schematic diagram illustrating modelling of a single axis (y-axis) with a control scheme in an exemplary embodiment.

FIG. 16 is a schematic diagram illustrating two interaction port behaviours with 2 DOF axes in an exemplary embodiment.

FIG. 17 is a schematic control block diagram of an admittance motion control loop for an individual translational joint in an exemplary embodiment.

FIG. 18 is a schematic diagram showing an overview of out-of-plane motion tracking framework, including pre-scan and visual servoing stages in an exemplary embodiment.

FIG. 19 is a schematic diagram of a proposed position-based admittance control scheme used to control a contact force between a probe and a body in an exemplary embodiment.

FIG. 20A is a perspective external view drawing of a needle insertion device (NID) in an exemplary embodiment.

FIG. 20B is a perspective internal view drawing of the NID in the exemplary embodiment.

FIG. 20C is a perspective view drawing of the NID having mounted thereon a needle in an angled orientation in the exemplary embodiment.

FIG. 20D is a perspective view drawing of the NID having mounted thereon a needle in an upright orientation in the exemplary embodiment.

FIG. 20E is a perspective view drawing of an assembly of the NID with an ultrasound probe mount at a first angle in the exemplary embodiment.

FIG. 20F is a perspective view drawing of an assembly of the NID with the ultrasound probe mount at a second angle in the exemplary embodiment.

FIG. 21 is a schematic flowchart for illustrating a method for registering real-time intra-operative image data of a body to a model of the body in an exemplary embodiment.

FIG. 22 is a schematic drawing of a computer system suitable for implementing an exemplary embodiment.

### DETAILED DESCRIPTION

Exemplary, non-limiting embodiments may provide a method and system for registering real-time intra-operative image data of a body to a model of the body, and an apparatus for tracking a target in a body behind a surface using an intra-operative imaging device.

In various exemplary embodiments, the method, system, and apparatus may be used for or in support of diagnosis (e.g. biopsy) and/or treatment (e.g. stone removal, tumour ablation or removal etc.). Examples of stone treatment options may include the use of ultrasound, pneumatic, laser etc. Tumour treatment options may include but are not limited to, excision, radiofrequency, microwave, cryotherapy, high intensity focused ultrasound, radiotherapy, focal delivery of chemicals or cytotoxic agents.

In various exemplary embodiments, the body may refer to a bodily organ or structure which include but are not limited to a kidney, lung, liver, pancreas, spleen, stomach and the like. The target may refer to a feature of interest within or on the body, which include but are not limited to a stone, tumour, cyst, anatomical feature or structure of interest, and the like. The body may be located within a human or an animal. In various exemplary embodiments, registration involves bringing pre-operative data (e.g. patient's images or models of anatomical structures obtained from these images and treatment plan etc.) and intra-operative data (e.g. patient's images, positions of tools, radiation fields, etc.) into the same coordinate frame. The pre-operative data and intra-operative data may be multi-dimensional e.g. two-dimensional (2D), three-dimensional (3D), four-dimensional (4D) etc. The pre-operative data and intra-operative data may be of the same dimension or of different dimension.

### 1. Method for Registering Real-Time Intra-Operative Data

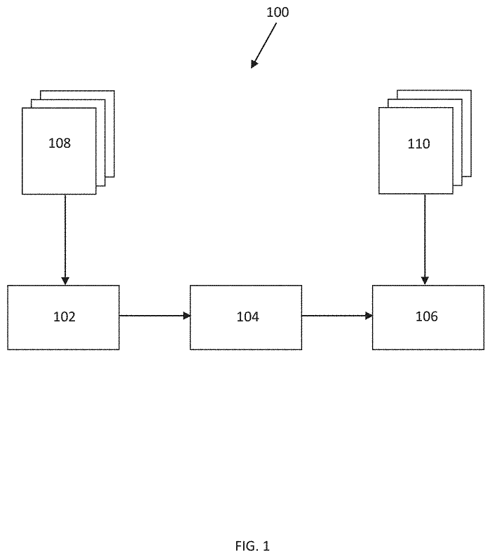

FIG. 1 is a schematic flowchart for illustrating a process 100 for registering real-time intra-operative image data of a body to a model of the body in an exemplary embodiment. The process 100 comprises a segmentation step 102, a modelling step 104, and a registration step 106.

In the segmentation step 102, a plurality of image data 108 of the body of a subject (e.g. patient) is segmented to delineate boundaries (e.g. lines, curves etc.) of anatomical features/structures on the plurality of image data 108. In general, image segmentation is a process of assigning a label to every pixel in an image such that pixels with the same label share certain characteristics. The plurality of image data 108 may be obtained pre-operatively and include but are not limited to computed tomography (CT) image data, magnetic resonance (MR) image data, ultrasound (US) image data and the like. The delineation of boundaries may be configured to be semi-automated or fully automated. The anatomical features/structures may include but are not limited to organs e.g. kidney, liver, lungs, gall bladder, pancreas etc., tissues e.g. skin, muscle, bone, ligament, tendon etc. growths e.g. stones, tumours etc.

In the modelling step 104, the segmented plurality of image data 108 of the body is used to construct/generate a model e.g. 3D model. The model may be a static or a dynamic model. For example, the model may be a static 3D model constructed from a plurality of two-dimensional (2D) image data. In another example, the model may be a dynamic 3D model which includes time and motion. Such a dynamic 3D model may be constructed from e.g. 4D X-ray CT image data (i.e. geometrically three dimensional with the 4^{th} dimension being time). In the exemplary embodiment, the modelling step 104 may comprise geometrization of the segmented plurality of image data 108 into a model, localisation of landmarks on the model, and rendering of the model for visualisation.

In the registration step 106, real-time intra-operative image data 110 of the body is used to register with the model of the body obtained from the modelling step 104. The real-time image data 110 may include but are not limited to CT fluoroscopy image data, real-time MR image data, real-time US image data and the like. In the exemplary embodiment, a registration algorithm e.g. modified affine registration algorithm is implemented to place one or more landmark features on the real-time intra-operative image data 110 and register each of the one or more landmark features to a corresponding landmark feature on the model.

In the exemplary embodiment, landmarks may be identified manually in both reconstructed models e.g. 3D models as well as real-time intra-operative image data to initiate and accelerate the registration process.

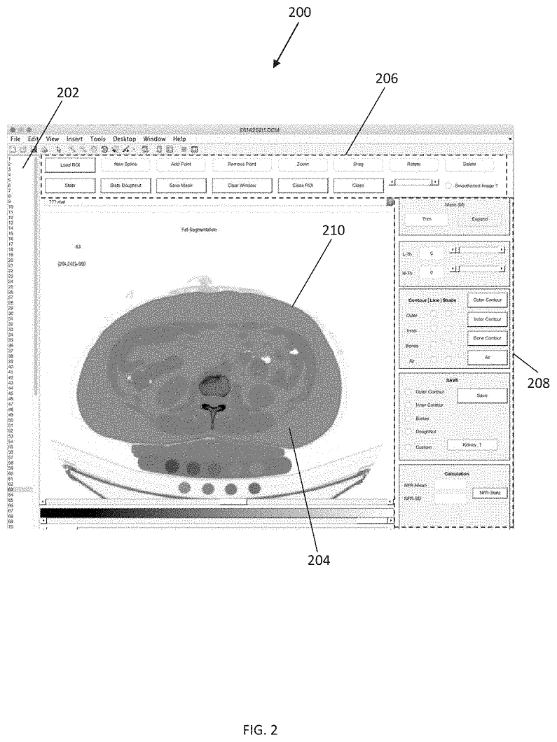

FIG. 2 is a screenshot of a graphical user interface (GUI) 200 of a customised tool for performing interactive segmentation (compare 102 of FIG. 1) of a plurality of image data (compare 108 of FIG. 1) in an exemplary embodiment. The GUI 200 comprises a left side panel 202 for displaying a list/library of image data of a body of interest e.g. cross-sectional view of a torso 204 of a subject, a top panel 206 comprising buttons associated with various functionalities such as addition/removal and manoeuvring of point(s), curve(s)/spline(s), and a right side panel 208 comprising buttons and sliders associated with other functionalities such as trimming and expanding of mask, adjusting of contours, saving the image data, and performing calculations. The plurality of image data may be image data obtained using imaging modalities/devices such as computed tomography, ultrasound or magnetic resonance etc.

Segmentation may be based on the concept that image intensities and boundaries of each tissue vary significantly. Initial segmentation may be based on a seeding and a region growing algorithm e.g. neighbourhood connected region growing algorithm. In one exemplary embodiment, the algorithm starts with manual seeding of some points in the desired tissue regions e.g. fat, bone, organ etc. Subsequently, the algorithm takes over and iteratively segments various tissues found on an image by pooling neighbourhood voxels which share similar pixel intensities (based on pre-defined intensity threshold ranges for different tissues). The algorithms may require manual intervention to adjust some parts of the boundaries at the end of the segmentation process to obtain good quality segmentation.

In the exemplary embodiment, the GUI 200 may be configured to perform segmentation of a plurality of image data to allow semi-automated boundary delineation (of outer skin, fat, and organ regions e.g. kidney of a subject) before manual correction to adjust the boundaries. The process involves manual seeding, multi-level thresholding, bounded loop identification, smoothening of boundaries, and manual correction.

It is recognised that the boundary of a target organ, e.g. kidney tissue, may be unclear on the plurality of image data captured by the pre-operative imaging device because of e.g., movement and over-processing by the algorithm. It will be appreciated that breathing movement of the subject (e.g. patient) and the orientation of the patient relative to the imaging capture device define the direction of movement of the target organ. If the direction of movement and the longitudinal axis of the target organ are not aligned, image artefacts may be generated, leading to unclear boundaries.

As for over-processing, the algorithm which approximates the boundary with pre-processing which may be excessive. For example, the algorithm may perform segmentation by flooding to collect pixels with the same intensity within the boundary. This may lead to leakage as additional voxels which are not part of the target tissue are also being segmented as being part of the target tissue.

It is recognised that the above issues may impact downstream geometry processing and therefore, it may be advantageous for segmentation to be semi-automatic (i.e. with manual intervention). In some exemplary embodiments, a stage-gate may be put in place to allow a user to verify the segmentation and make adjustment (if any), before proceeding further with the downstream processing.

It is also recognised that variations in image intensities and boundaries of each tissue may impact the automation of segmentation. To reduce computational cost, customised image pre-processing routines which may be used for segmentation of different tissues (e.g. outer and inner boundaries of the skin, fat, bone, and organ e.g. kidney) are created. Such customised image pre-processing routines may be pre-loaded into the customised tool of the exemplary embodiment.

It would be appreciated that while the core algorithm or method may be similar, segmentation of image data from different sources may involve variations in the parameters, in the level of pre-processing before applying the segmentation, and in the level of manual intervention. For example, when the customised tool is used to segment MR images, the seeding points and threshold values/coefficient may need to be adjusted based on the range of pixel intensities and histogram. In addition, the contrast-to-noise ratio (CNR) may vary with different imaging modalities and thus the amount of manual adjustment/correction to delineate boundaries may differ between imaging modalities.

In the exemplary embodiment, the plurality of image data are CT images obtained using computed tomography. The data is pre-processed with windowing (i.e. by selecting the region where the body of interest e.g. kidney would be, right or left side of the spine, lines to define above-below regions to narrow down the search). Anisotropic diffusion filtering is then applied to reduce the noise while preserving the boundary. In the exemplary embodiment, the threshold values for segmentation is set at between 100 to 300 HUs (Hounsfield unit) and manual seeding is done by selecting a pixel in the kidney region to accelerate the segmentation process.

In exemplary embodiments, segmentation may be performed sequentially to reduce manual correction, implement tissue-specific segmentation routines, and achieve computational efficiency. For example, the outer boundary of the skin 210 may be segmented first to eliminate all outer pixels from the search for other tissues, followed by the inner boundary of the skin 210, and then the search for bone regions and voxels indices to narrow down the search region for segmenting organ regions e.g. kidney.

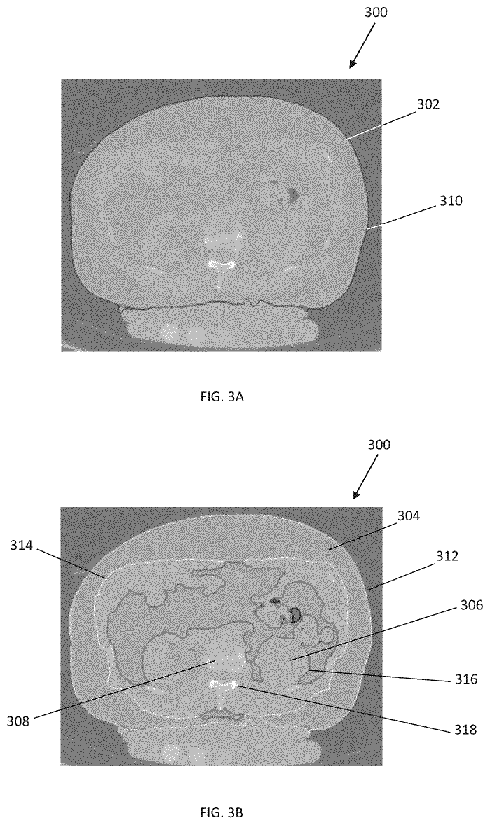

FIG. 3A is a processed CT image 300 of a subject with a first segmentation view in an exemplary embodiment. FIG. 3B is the processed CT image 300 of the subject with a second segmentation view in the exemplary embodiment. The processed CT image 300 represents a sample output of an initial segmentation with various boundaries depicting outer boundary 310 of the skin 302, inner boundary 312 of the skin 302, boundary 314 of the fat region 304, boundary 316 of the kidney region 306, and boundary 318 of the bone region 308, before manual corrections. As shown, these boundaries are outputted as curves for further processing. Masks are also kept with the processed images in case there is a need for reprocessing of the images.

In various exemplary embodiments, after a plurality of image data of a subject is segmented, the plurality of segmented image data is further subjected to modelling (compare 104 of FIG. 1) which may comprise geometrization of the segmented plurality of image data into a model, localisation of landmarks on the model, and rendering of the model for visualisation.

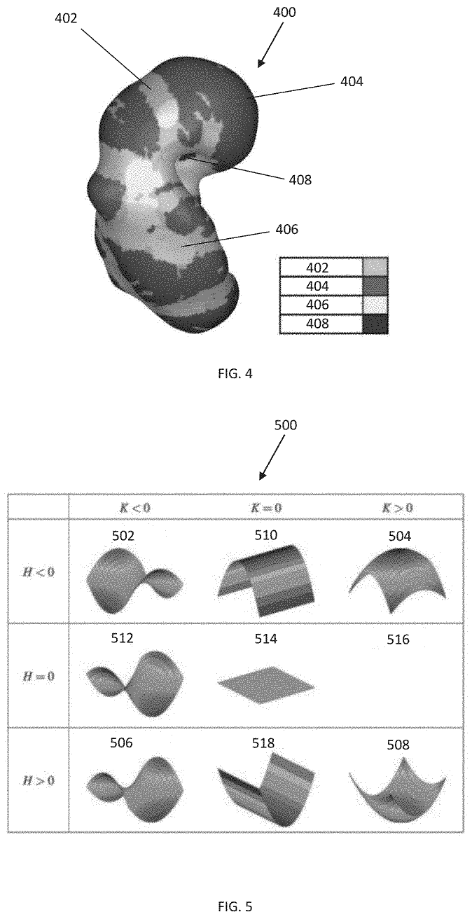

FIG. 4 is a 3D model of a kidney 400 in an exemplary embodiment. It would be appreciated that the model is based on the body or region of interest. In other exemplary embodiments, the model may be of other organs e.g. lung, liver, pancreas, spleen, stomach and the like.

The 3D model of the kidney 400 is constructed from a plurality of image data e.g. CT image data which has undergone segmentation to delineate the boundaries of regions of tissues e.g. bone, fats, skin, kidney etc. The segmentations in the plurality of CT image data may be smoothened with a 3D Gaussian kernel. Depending upon the need/requirement, different kinds of algorithms may be used to generate a polygonal e.g. triangular or quadrilateral mesh for visualisation. For example, the algorithm may be implemented with a simple triangulation based on a uniform sampling of curves using circumference of the curves as reference (i.e. cloud points-based computation). In another example, the algorithm may be a marching cubes algorithm to generate fine mesh and this second algorithm may require a higher computational cost as compared to the simple triangulation. The generated triangulated meshes are then used to render reconstructed 3D anatomical models for visualisation and downstream intra-operative image registration to real-time image data taken using an intra-operative imaging device/modality e.g. ultrasound.

In the exemplary embodiment, the 3D model of the kidney 400 is constructed using simple triangulation. Simple triangulation is chosen to reduce the computational power needed to apply a transformation matrix and visualise the model in real-time. Even though the simple triangulation from the cloud points generated by boundary delineation may generate triangles with uneven areas, the goal of the exemplary system is to allow the kidney to be visualised and displayed for a user, thereby allowing coordinates of the affected tissue to be identified. Therefore, while computationally expensive marching cube algorithm may generate fine-triangles with better visualisation, it may not be as fast to be suitable for use in real time. In the case of pre-operative visualisation in a stand-alone system, the marching cube-based visualisation may be used to study the affected tissue as well as the kidney model due to its better visualisation.

In the exemplary embodiment, segmentations and 3D triangular mesh of objects/bodies/regions of interest are individually labelled instead of merging them as a single giant mesh. This advantageously lowers the computational cost and enables a user to interactively visualise them. For the kidney model 400, soft tissues such as the ureter and renal vein are segmented approximately as computed tomography may not be an ideal imaging modality to quantify these soft tissues. Approximate models of the soft tissues are created for landmarks localisation and visualisation purposes. These soft tissues are modelled as independent objects; and superimposed over the kidney model. The modelling methods may be implemented on a suitable computing environment capable of handling the computational workload. It would be appreciated that when implemented in a MATLAB® environment, the rendering speed may be slightly slower, even with a 16 GB RAM workstation due to the large number of triangles.

As for landmark localisation, one or more landmark features may be identified and labelled on the model for subsequent use in a registration step (compare 106 of FIG. 1). For example, the one or more landmark features may be prominent surface points/landmarks or measurements between prominent points of the body (i.e. kidney). For example, the central line drawn by connecting the superior-most and inferior-most points/poles of the kidney may be used as one of the landmarks. In this case, the line drawn may be representative of the distance between the superior-most and inferior-most points of the kidney. Subsequently, a list of feature points of the kidney model for registration is generated using curvature measurement techniques. This concept of using curvature measurement techniques is implemented in order to reduce the number of landmarks needed to register the model with intra-operative images e.g. ultrasound images. In some cases, the intra-operative image resolution e.g. ultrasound image resolution may not be sufficient to generate a similar level of feature points like the 3D model. This may be overcome by using the most prominent surface landmarks using a combination of Gaussian and mean curvatures. As shown in FIG. 4, the 3D model of the kidney 400 comprises saddle ridge 402, peak 404, saddle valley 406 and pit 408 landmarks. It would be appreciated that the one or more landmark features may include other points/landmarks such as the longitudinal and lateral axes of the body (i.e. kidney), Minkowski space geometric features in high dimension space, outline of the kidney, and calyces (upper, middle, or lower) of the kidney.

FIG. 5 is a set of images 500 showing different curvature types by sign, in Gaussian and mean curvatures. Principal curvatures on the triangular mesh are calculated for each vertex of a body (e.g. kidney) using a local surface approximation method. The principal curvatures and their corresponding principal directions represent the maximum and minimum curvatures at a vertex. From these principal curvatures, the Gaussian and mean curvatures are calculated, and changes in their signs are used to identify shape characteristics for deciding landmarks as shown in FIG. 5. Gaussian and mean curvatures and their signs together depict different surface characteristics of a model e.g. kidney model (after smoothening of the mesh). In the context of discrete meshes, only 4 types of landmarks (i.e. saddle ridge 502, peak 504, saddle valley 506 and pit 508) are identified. These identified landmarks regions may be seeded and labelled interactively to start a registration process (compare 106 of FIG. 1). The other landmarks shown on FIG. 5 include ridge 510, minimal 512, flat 514, impossible (i.e. no landmark) 516, and valley 518.

In various exemplary embodiments, a model is generated/constructed from a plurality of image data e.g. images obtained using a pre-operative imaging device/modality. The model may be used in a registration step (compare 106 of FIG. 1) which may comprise labelling/localisation of landmarks on real-time image data and registration of the labelled real-time image data to the model.

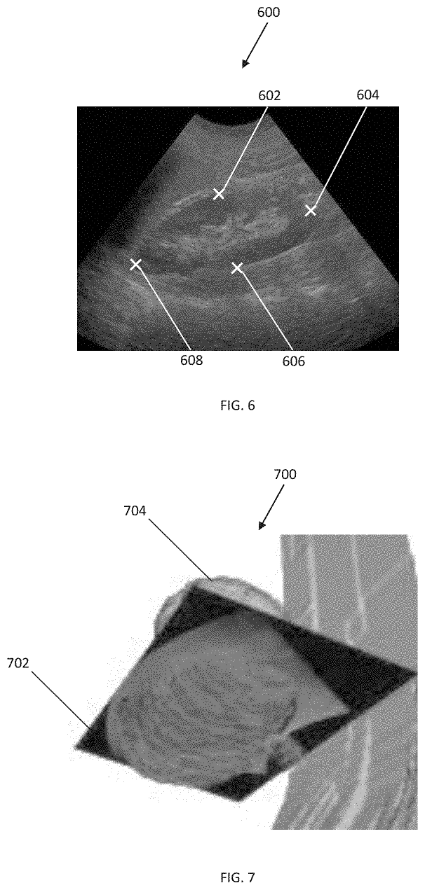

FIG. 6 is an ultrasound image 600 labelled with a plurality of landmarks 602, 604, 606, 608 in an exemplary embodiment. FIG. 7 is a composite image 700 showing a 2D ultrasound image 702 and 3D reconstructed model 704 of a kidney after affine 3D-2D registration in an exemplary embodiment.

In the exemplary embodiment, landmarks are used as initial registration points in order to simplify the registration work flow and also to reduce computational workload. In various exemplary embodiments, sub-sampling or down-sampling of the model may be performed to match the resolution of an intra-operative imaging device. In the exemplary embodiment, the 3D reconstructed model is sub-sampled to match the resolution of ultrasound images.

In use, a user (e.g. surgeon) positions an imaging probe (e.g. ultrasound probe) over a region of interest (e.g. kidney) of a subject (e.g. patient). The ultrasound probe may be in contact with the skin surface of the patient above the kidney region. A real-time ultrasound image 600 of the kidney is obtained by the ultrasound probe and is displayed on an image feedback unit having a display screen. The surgeon adjusts the position of the ultrasound probe to locate a suitable image section of the kidney. Once a suitable image section of the kidney is located, the surgeon interactively selects/labels one or more landmark features e.g. 602, 604, 606, 608 on the ultrasound image 600 and the one or more landmarks are highlighted by the image feedback unit on the display screen. The ultrasound image 600 with the one or more labelled landmarks e.g. 602, 604, 606, 608 are processed using a registration module which executes a registration algorithm/method (e.g. affine 3D-2D registration) to match the one or more labelled landmarks on the ultrasound image to corresponding landmarks labelled in the model e.g. 3D reconstructed model 704. Rendering of the 3D reconstructed model 704 is performed to project the corresponding landmarks on the 3D model on a 2D plane to facilitate registration to the one or more labelled landmarks on the ultrasound image. The result is the composite image 700 showing the 2D ultrasound image 702 and 3D reconstructed model 704, thereby allowing the kidney to be visualised and displayed for a user, and allowing coordinates of the affected tissue and kidney stone to be identified.

In the exemplary embodiment, to perform registration of real-time images to a model constructed using pre-operative images, the following assumptions are made. First, it is assumed that pre-operative planning images as well as real-time images are acquired with similar subject e.g. patient positioning (e.g. prone position—face down). This is different from routine diagnostic imaging procedures, where pre-operative images are acquired in supine position (face-up) but the biopsy procedure is performed in prone position for easy accessibility. Second, it is assumed that a patient's breathing pattern does not change to a level that would affect the movement pattern of the body e.g. kidney. Third, the size and shape of the body e.g. kidney is assumed to not shrink/swell significantly from the time pre-operative images were taken.

Based on the above assumptions, the superior-most (based on a pre-defined coordinate system) and the inferior-most points of the body e.g. kidney can be geometrically classified and identified as respective “peaks” (compare 504 of FIG. 5) due to their unique shape independent of the orientation of the kidney. A user interactively places the superior-most and inferior-most points on a suitable real-time mid-slice image of the kidney (e.g. a sagittal or frontal plane image of the kidney showing both the superior-most and inferior-most points on the same image) to initiate the registration process. These two points are tracked in real-time by simplifying the kidney at the particular slice as an oval shape object by fitting (using axes ratio of 1.5 in 2D). While it is assumed the patient positioning during the pre-operative and intra-operative imaging are similar, some misalignment between the model and real time images may be expected. The landmarks identified on the 3D model are projected to a 2D plane to register with the selected landmark data points on the real time image, and in turn, making the process computationally efficient. Registration is done by minimizing the mean square error between the 3D model and the selected landmarks data points (due to some misalignment between the model and real time images, the distance between the landmarks on the model and real-time image is not zero). Once a transformation coordinate matrix is calculated, the matrix is applied to the real-time image to visualize both 3D model and the image as shown in FIG. 7. The same matrix will be used to reference the position of the affected tissue for biopsy.

In exemplary embodiments, a subject's e.g. patient's respiration is taken into consideration when registering 3D volume with 2D ultrasound images. Due to movement of the organ (e.g. during respiration), the images acquired by the ultrasound tend to have motion artefacts. These artefacts affect the clear delineation of the boundaries. Therefore, once initial segmentation is performed, manual intervention by a user is needed to verify and correct any error in those delineated boundaries (slice-by-slice). In various exemplary embodiments, a system for performing registration comprises an interactive placing feature which allows the user to perform such a manual intervention function. In addition, the interactive placing feature allows the user to manually click/select a pixel on the real-time image to select a landmark.

For the purposes of algorithm testing, virtually simulated ultrasound images are used for registering to CT images. The virtually simulated ultrasound images are made to oscillate with a sinusoidal rhythm to mimic respiration of a subject e.g. patient. It would be appreciated that in real-life scenarios, respiration of patients may change due to tense moments such as when performing the biopsy or simply being in the operating theatre. Adjustments to the algorithm may be required with registration of real-life CT/MR images and 3D US images of the same subject.

In the exemplary embodiment, a modified affine registration algorithm is implemented by interactively placing landmarks on US images and registering the landmarks to the corresponding one on the 3D geometric models. Affine 3D-2D registration method iteratively aligns the 3D models (which comprise cloud of points and landmarks on the mesh) to the landmarks on the US images by minimizing the Euclidean distance between those landmarks or reference points. To speed up the registration process, two additional landmarks may be used, i.e. the most superior and inferior points/poles of the kidney. These additional landmarks assist in quickly assessing the initial transformation for further subsequent fine-tuning. This method is useful for realignment when the FOV (field of view) goes out of the kidney, assuming the transducer orientation does not change. An option may be also provided to allow the landmarks to be re-selected/identified in case of a complete mismatch. In the exemplary embodiment, the landmarks are selected at the maximum exhalation position and then tracked to quantify the respiration frequency as well. In exemplary embodiments, the landmarks are selected at the maximum exhalation position, and other stages of respiration are ignored. In other words, the landmarks are selected at substantially the same point in a respiratory cycle.

It would be appreciated that the 3D reconstructed model is based on the body or region of interest. In other exemplary embodiments, the model may be of other organs e.g. lung, liver, pancreas, spleen, stomach and the like. It would also be appreciated that any real-time imaging modality can be used for image registration as long as the required customisation of the proposed system is done. For example, real-time MRI is possible only with low image quality or low temporal resolution due to time-consuming scanning of k-space. Real-time fluoroscopy can also be used.

### 2. Apparatus/Robot for Tracking a Target in a Body Behind a Surface Using an Intra-Operative Imaging Device

It would be appreciated that in various exemplary embodiments, the method and system for registering real-time intra-operative image data of a body to a model of the body may be applied in a wide range of surgical procedures like kidney, heart and lung related procedures. For the purposes of illustration, the method and system for registering real-time intra-operative image data of a body to a model of the body are described in the following exemplary embodiments with respect to a percutaneous nephrolithotomy (PCNL) procedure for renal stone removal.

Percutaneous nephrolithotomy (PCNL) is a minimally invasive surgical procedure for renal stone removal and the benefits of PCNL are widely acknowledged. Typically, PCNL is a keyhole surgery that is performed through a 1 cm incision under ultrasound and fluoroscopy guidance. Clinical studies have shown that PCNL procedure is better than open surgery due to shortening in the length of hospital stay, less morbidity, less pain and better preservation of renal function. In addition, studies have shown that PCNL is able to achieve higher stone free rates. Hence, PCNL surgery is widely acknowledged over traditional open surgery for large kidney stone removal.

However, planning and successful execution of the initial access to the calyces of the kidney is challenging due to respiratory movement of the kidney and involuntary motion of the surgeon's hand. To make things more complicated, the surgeon needs to take control of several other surgical instruments simultaneously. Existing PCNL procedures rely heavily on manual control. Hence, the ability to gain access to the target depends heavily on operator's experience, judgement and dexterity. Several needle punctures are often required for successful percutaneous access which increases the risk of bleeding and other forms of damage to the nearby organs, e.g. renal bleeding, splanchnic, vascular and pulmonary injury. Despite the advancements in image-guided surgical robots, the involuntary motion compensation of both patient and surgeon during PCNL surgery remains a challenge. Further, PCNL is traditionally performed with the aid of X-rays fluoroscopy, which exposes both patient and surgeon to harmful radiation.

The above problems associated with PCNL have been identified and an apparatus/robot for tracking a target in a body behind a surface using an intra-operative imaging device has been developed. This apparatus may be used in conjunction with the afore-mentioned registration process.

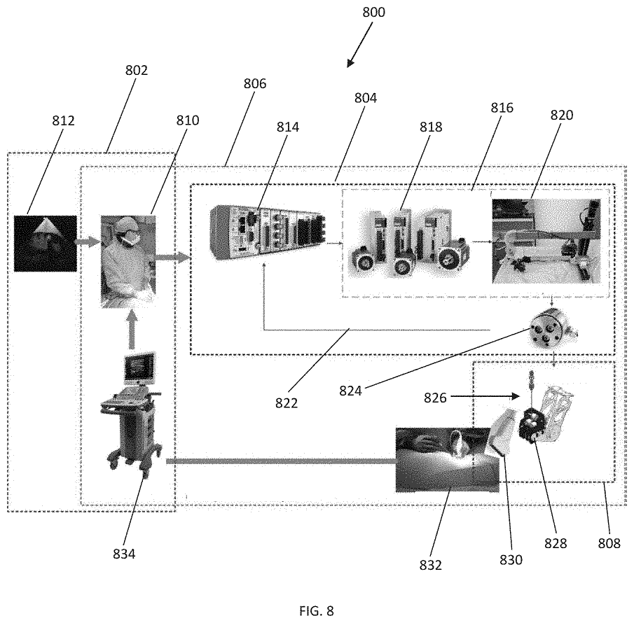

FIG. 8 is a schematic diagram of an overview of a system 800 for implementing a method for tracking a target in a body behind a surface using an intra-operative imaging device in an exemplary embodiment. The system 800 comprises an image registration component 802 for registering real-time intra-operative images to a model, a robot control component 804 for providing motion and force control, a visual servoing component 806, and a needle insertion component 808. In the image registration component 802, real-time intra-operative image data of a body is registered to a model of the body (compare 100 of FIG. 1). A surgeon 810 uses an intra-operative imaging device e.g. ultrasound imaging to obtain an ultrasound image 812 of a target kidney stone and calyces for PCNL surgery. The ultrasound image 812 is registered to a model constructed using pre-operative images e.g. a plurality of CT images.

In the robot control component 804, a robot having force and motion control is operated by the surgeon 810. The robot may provide 6 degrees of freedom (DOF) motion and force feedback. The robot comprises a mechatronics controller 814 which provides motion control 816 using motors and drivers 818 for moving a manipulator 820. The manipulator 820 provides force control 822 via force sensors 824 back to the mechatronics controller 814.

In the needle insertion component 808, needle insertion is performed by the robot at its end effector 826. The end effector 826 comprises a needle insertion device 828 and an imaging probe e.g. ultrasound probe 830. The end effector 826 is configured to contact a patient 832 at his external skin surface. The visual servoing component 806 comprises an image feedback unit 834 which is used to provide real-time images obtained by the imaging probe 830 and the robot relies on such information to provide out-of-plane motion compensation.

The system 800 for tracking a target in a body behind a surface using an intra-operative imaging device may be an apparatus/robot which has the following features: (1) a stabilizing manipulator, (2) ultrasound-guided visual servoing for involuntary motion compensation, (3) 3-D reconstruction of an anatomical model of the kidney and stone from CT images, and ultrasound-based intra-operative guidance, and (4) automatic needle insertion. The stabilizing manipulator may address the problem with unintended physiological movement while at the same allow the user to handling multiple tasks at the same time. The manipulator may be placed on a mobile platform that can be pushed near to the patient when required, so as to anticipate potential issues of space constraint due to an additional manipulator in the surgical theatre. The ultrasound image-guided visual servoing method/mechanism described herein may provide tracking out-of-plane motion of the kidney stones influenced by the respiratory movement of the patient during PCNL surgery. In addition, an admittance control algorithm is proposed to maintain appropriate contact force between ultrasound probe and the patient's body when the operator releases the probe after initial manual positioning. This not only provides better image quality but also reduces burden on the surgeon so that he can concentrate on the more critical components.

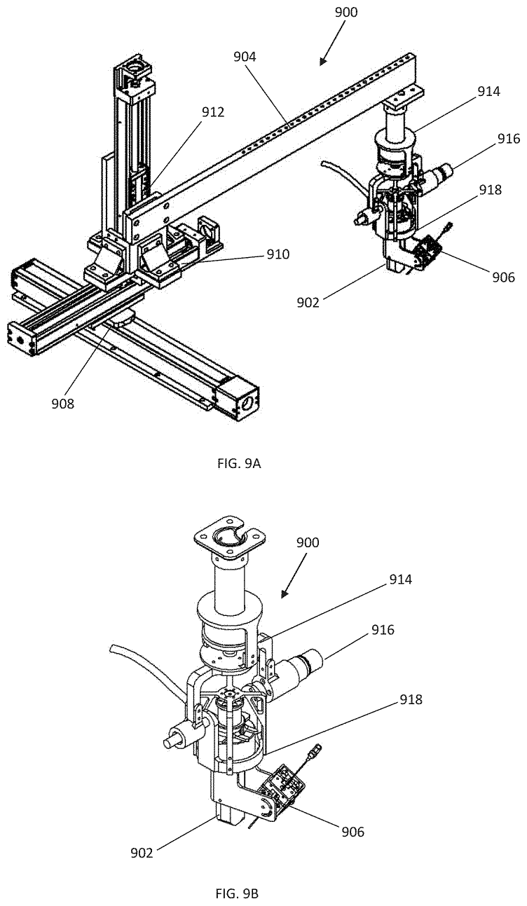

FIG. 9A is a perspective view drawing of a robot 900 for tracking a target in a body behind a surface using an intra-operative imaging device in an exemplary embodiment. FIG. 9B is an enlarged perspective view drawing of an end effector of the robot 900 in the exemplary embodiment. The robot 900 comprises an imaging probe 902 for performing scans of the body, a manipulator 904 for engaging and manipulating the imaging probe 902 coupled to its end effector, and a needle insert device e.g. needle driver 906 coupled to the manipulator 904 at the end effector. The manipulator 904 may comprise one or more joints e.g. translational joints 908, 910, 912, and rotational 914, 916, 918 to provide 6-DOF (degree of freedom) for a user e.g. surgeon to move the end-effector of the robot 900. The needle insert device 906 may comprise holding means for holding a needle at an angle directed at the target e.g. stones in the body e.g. kidney. The imaging probe 902 may be coupled to an image feedback unit (compare 834 of FIG. 8) for providing real-time intra-operative image data of the scans obtained by the imaging probe 902.

The robot 900 may further comprise a control unit (not shown) for positioning the probe by controlling the manipulator. The control unit may comprise an image processing module and a registration module. The image processing module may be configured to perform segmentation and modelling (compare 102 and 104 of FIG. 1) of a plurality of image data obtained using a pre-operative imaging device. In other words, the image processing module may be configured to segment a plurality of image data of the body obtained using a pre-operative imaging device; construct a model of the body from the segmented plurality of image data, said model comprising an optimal needle trajectory information, and said optimal needle trajectory information comprising positional information on a point on the surface and a point of the target; and identify one or more landmark features on the model of the body. The registration module may be configured to perform registration (compare 106 of FIG. 1) of the real-time intra-operative image data of the body to the model of the body by matching one or more landmark features labelled on the real-time intra-operative image data to one or more corresponding landmark features on the model of the body.

In the exemplary embodiment, the manipulator 904 is configured to directly manipulate the imaging probe 902 in collaboration with the control unit such that the needle substantially follows the optimal needle trajectory information to access the target in the body.

In use, a user e.g. surgeon manipulates the end effector of the manipulator 904 having the imaging probe 902 and needle insert device 906 coupled thereto. The robot 900 collaborates with or adjusts the force/torque applied by the surgeon and moves the end effector accordingly. The surgeon then selects the targeted region e.g. kidney so that 3-D registration between the intra-operative images and pre-operative images e.g. CT images is performed. Once the needle is determined to be positioned at the correct location, the surgeon activates the needle driver 906, by e.g., pushing a button which controls the needle driving process. The robot 900 then drives the needle into the target e.g. stone. In an alternative exemplary embodiment, instead of using pre-operative images e.g. CT images to register with the intra-operative images, pre-scanning of US images may be performed to create a 3D volume information of the targeted region for subsequent registration with intra-operative images.

### 2.1.1 Collaborative Stabilising Manipulator—Concept of Interaction Port Behaviour

In various exemplary embodiments, a manipulator (compare 904 of FIG. 9) may be a collaborative stabilizing manipulator. The manipulator may be designed based on a phenomenon known as two interaction port behaviours which may be relevant to surgical procedures e.g. PCNL. The concept of interaction port behaviours may be described as behaviour which is unaffected by contact and interaction. Physical interaction control refers to regulation of the robot's dynamic behaviour at its ports of interaction with the environment or objects. The terminology “collaborative control” has a similar meaning with physical human-robot interaction (pHRI) (which is also referred to as cooperation work). The robot interacts with the objects and the control regulates the physical contacted interaction. In PCNL, the surgeon plays a dominant role, guiding the robot to target the initial access for a better needle puncture. The human has physical contacts with the robot. On the other hand, the patient is considered as an interactive environment to the robot. Emphasis is placed on control design when handling interaction port behaviour. Control schemes may be separated into two parts—rotational and translational parts.

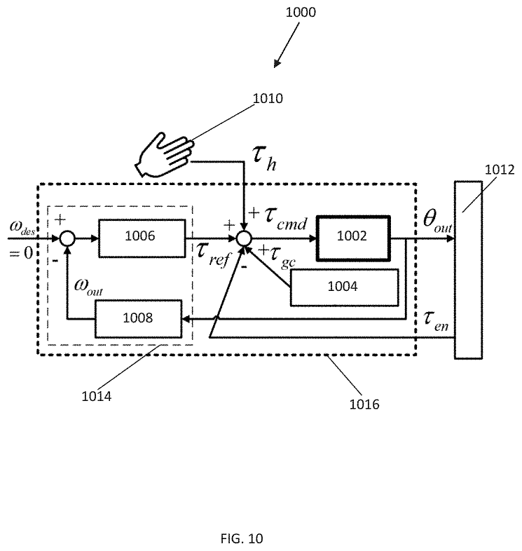

FIG. 10 is a schematic diagram of a control scheme 1000 for rotational joints of a manipulator in a robot in an exemplary embodiment. The control scheme 1000 may apply to rotational joints 914, 916 and 918 of the manipulator 904 in FIG. 9 to impart back-drivable property without torque sensing. The control scheme 1000 comprises a motor 1002, a gravity compensator 1004, a velocity controller 1006, and a velocity estimator 1008. The motor 1002 receives an input signal T_{cmd} which is a summation of signals from the gravity compensator 1004 (represented by T_{gc}), velocity controller 1006 (represented by T_{ref}), an interactive torque from a user e.g. surgeon 1010 (represented by Tₕ), and a negative feedback signal from an interactive environment e.g. human subject/patient 1012 (represented by Tₑₙ). The motor 1002 produces a position output θₒᵤₜ to the patient 1012. In the exemplary embodiment, control scheme 1000 comprises multiple feedback loops. The patient 1012 provides a negative feedback and the gravity compensator 1004 provides a positive feedback to the motor 1002. At the same time, velocity estimator 1008 provides an output velocity ωₒᵤₜ as negative feedback to the velocity controller 1006, and the output of the velocity controller is provided to the motor 1002.

For the rotational motors 1002 in the rotational joints of the manipulator, only the velocity controller 1004 is designed as they are all back drivable with light weights, as shown in FIG. 10. The interactive force from the user e.g. surgeon 1010 may be considered as the driving force for the robot. The velocity controller 1006 with a velocity estimator 1008 and a gravity compensator 1004 are designed. By setting desired velocity ω_{des} as zero and adjusting the bandwidth of the closed-loop transfer function for the control scheme 1000, the position output, θₒᵤₜ, can be regulated for interactive torque, Tₕ.

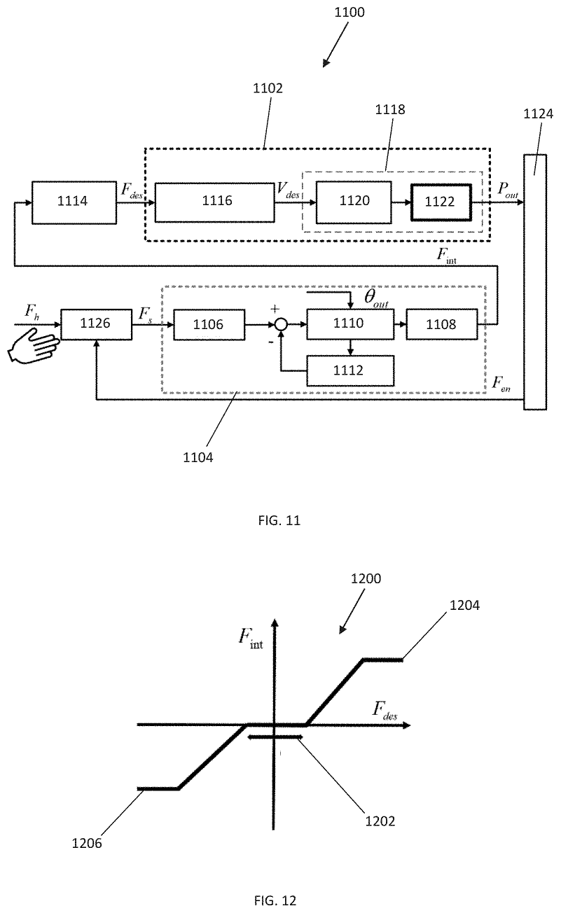

FIG. 11 is a schematic diagram of a control scheme 1100 for translational joints of a manipulator in a robot in an exemplary embodiment. The control scheme 1100 may apply to translational joints 908, 910 and 912 of the manipulator 904 in FIG. 9 to impart variable impedance control with force signal processing. In the control scheme 1100, a variable admittance motion control loop 1102 with force signal processing is used for the bulky translational linear actuators, i.e. joints 908, 910 and 912. To obtain a clean interactive force, Fᵢₙₜ, with correct orientation, the force/torque signal pre-processing 1104 comprises a low pass filter 1106, a high pass filter 1108, a 3D Euler rotational matrix 1110 which receives an angle output θₒᵤₜ from an individual motor, and instrument weight compensation 1112 to provide compensation in case of extra-measurement of the force. In addition, dead zone and saturation filters 1114 are employed to compensate for noise in the force feedback and to saturate the desired force at an upper limit (to improve the control of a relatively large force). FIG. 12 is a graph 1200 of interactive force, Fᵢₙₜ against desired force, F_{des} and showing regions of dead zone 1202, positive saturation 1204 and negative saturation 1206 in an exemplary embodiment. The desired force, F_{des}, is the control input for the robotics system which comprises a variable admittance control 1116 for providing the desired velocity input V_{des} to a velocity-controlled system 1118. The velocity-controlled system 1118 comprises a velocity controller 1120 and a motor 1122. The motor 1122 provides an output position Pₒᵤₜ of the end effector on a patient 1124. The patient 1124 provides/exerts a reaction force Fₑₙ back on the end effector, which is detected by a force/torque (F/T) sensor 1126 which then moderates the input force signal Fₛ (force sensed by sensor 1126) to be fed into the force/torque signal pre-processing 1104. The force/torque (F/T) sensor 1126 is also configured to detect force Fie, exerted by a hand of a user. The translational parts of the robot are designed with variable admittance and velocity control scheme. The controller behaves as admittance to regulate the force difference between desired force and the environment reaction force, Fₑₙ (FIG. 11), at the two interaction ports.

As shown in FIG. 10, for the rotational joints, a velocity controller of back-drivable rotational joints with zero desired force and velocity command is used. In addition, based on FIG. 11, a variable admittance control loop is used to regulate the interaction between the input force from the surgeon and the force experienced by the patient. The variable admittance motion control loop obtains force input signals which have been processed/filtered and outputs a desired command. More details about 6 DOF control scheme along with system identification are analysed and illustrated in the following exemplary embodiments.

### 2.1.2 Collaborative Stabilising Manipulator—Modelling and System Identification

In various exemplary embodiments, each of the individual axis of a joint (compare 908, 910, 912, 914, 916, 918 of FIG. 9) may be analysed by modelling. In a serial robot, uncertainty and dynamics are accumulated. In the exemplary embodiment, a decoupled structure is used and hence, the effect of accumulation is minimised. As such, the cross-axis uncertainty and dynamics between axes of a robot (compare 900 of FIG. 9) may be ignored due to the decoupled property of the structure for the robot which is unlike articulated arms Hence, once the model parameters are obtained by system identification, the control for each axis may be designed individually.

Both transfer functions (e.g., velocity and torque) of a single linear actuator of e.g., ball screw type and a DC motor may be derived as a first order model according to equation (1).

$$\{ \begin{matrix}\frac{{ω}_{o u t}}{{τ}_{c m d}} = \frac{1}{J s + B} , & rotational axes \\ \frac{{V}_{o u t}}{{τ}_{c m d}} = \frac{1}{M s + B} , & translational axes\end{matrix} \qquad ( 1 )$$

where M, J, B denote the mass, inertia and damping of the motor respectively. τ_{cmd} is the torque input command (Nm) and ωₒᵤₜ, Vₒᵤₜ are the angular velocity (rad/s) and velocity output (mm/s), respectively.

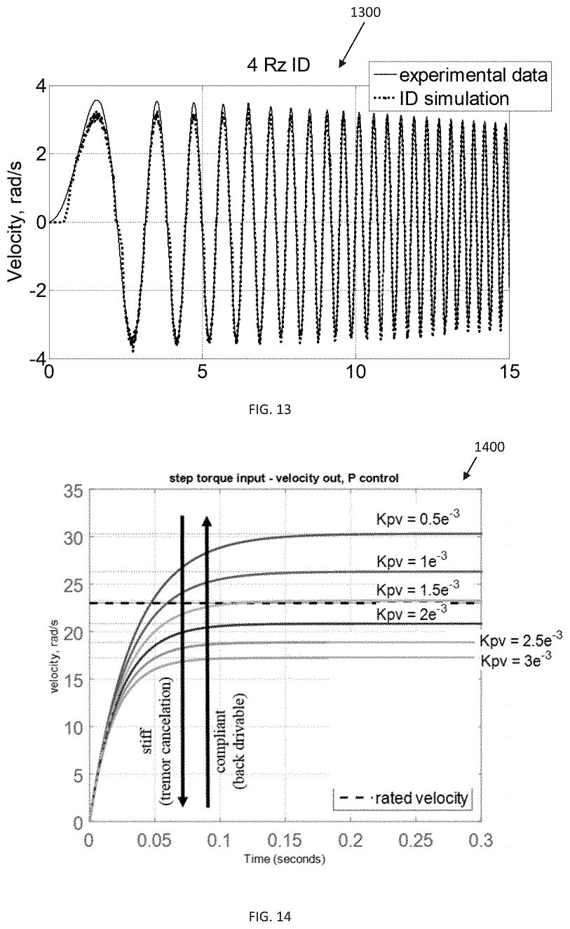

To obtain the parameters of the transfer functions in equation (1), a swept sine torque command, from low to high frequency, may be employed. The range of frequency is adjusted based on the natural frequency of each developed decoupled structure. The ratio of torque input and (angular) velocity output has been analysed using the system ID toolbox of MATLAB™. For example, the simulation for one of single axis (4^{th} R_{z}) is shown as FIG. 10. Region 1014 is the velocity output of the motor and region 1016 is the curve-fitting result. With the transfer functions in hand, the parameters for controllers in each axis can be designed. FIG. 13 is a graph 1300 of system identification for one single axis—swept sine velocity experimental data obtained from an exemplary embodiment implementing the designed controllers, in comparison with the simulated data.

### 2.1.3 Collaborative Stabilising Manipulator—Back Drivable Rotational Axis Control Scheme

To ensure the system is back-drivable while also stable, a modelling analysis and stability issue for rotational axes is described. For a single axis DC motor, a proportional velocity control with human torque, τₕ(s), and environment reaction, τₑₙ(s), without a gravity compensator is illustrated. The torque difference Δτ(s) at two interaction ports is defined as

Δτ(s)=τₕ(s)−τ(s) (2)

Assume that the velocity controller is G_{c}(s) and the motor transfer function is G(s), the closed-loop relation between the torque difference and angular velocity output, ωₒᵤₜ(s), can be described as follows,

$$\begin{matrix}\frac{{ω}_{out} ( s )}{Δ τ ( s )} = \frac{G ( s )}{1 + {G}_{c} ( s ) G ( s )} \\ = \frac{\frac{1}{J s + B}}{1 + {K}_{p v} \frac{1}{J s + B}} \\ = \frac{1}{J s + ( B + {K}_{p v} )}\end{matrix} \qquad ( 3 )$$

where Kₚᵥ represents the proportional velocity control gain. Consider the characteristic equation of (3), the zero is stable if

$$J s + ( B + {K}_{p v} ) = 0 \\ s = \frac{- ( B + {K}_{p v} )}{J} < 0. \qquad ( 4 )$$

In this case, J and B is the inertia and damping of the motors with positive values. Hence, Kₚᵥ can be any value that is greater than zero.

FIG. 14 is a graph 1400 showing stability and back-drivable analysis in an exemplary embodiment. In the graph 1400, the step torque is input to the system, resulting in an output velocity as shown in the graph. The change of velocity control with respect to different Kₚᵥ (the proportional velocity control gain) is shown. To determine the range of Kₚᵥ while still maintaining the stability, the rated velocity of the motor is considered with the control parameters. Δτ(s) is taken as disturbance to the closed-loop system. The meaning of back-drivable is that the system has less rejection for the disturbance. Therefore, a step torque command is sent into equation (3) (take 4^{th} R_{z} axis as the example) and the angular velocity output can be observed in FIG. 14. When the Kₚᵥ is getting larger, the speed is slower. In other words, the system rejects ΔT(s) to keep the control scheme working and become stiffer. This helps the performance as it has the capability to reduce human hand's tremor. However, another objective is also to ensure each of the rotational joint can at least achieve the rated speed by human motion. Therefore, the best trade-off for Kₚᵥ is the value which is closer to rated velocity (represented by a horizontal dotted line), in the case of FIG. 14, Kₚᵥ=0.0015.

For 5^{th} R_{y} axis, the gravity compensation is designed to hold the US probe. The gravity controller, τ_{gc}, is described according to equation (5) as follows,

τ_{gc}(θₒᵤₜ₅)=mᵤglᵤ(θₒᵤₜ₅) (5)

where mᵤ and lᵤ are the mass and half length of the instrument, respectively. Notation g is the gravity and θₒᵤₜ₅ is the 5^{th} rotational angle output. Besides, due to the high gear ratio (91:1) for 6^{th} Rₓ axis, the stiffness of the motor need not be increased. Thus, no control scheme is applied in this axis as it is already executed in a passive manner once it is powered.

2.1.4 Collaborative Stabilising Manipulator—Translational Axis with Variable Impedance Control

Next, the control schemes for 3 translational joints using variable impedance control are described. The dynamic model for these 3-DOF linear actuators is

M\ddot{q}+B\dot{q}+g=τ_{cmd} (6)

where q∈Rⁿ, with n=3, is the vector of translational joint variables, M∈R^{n×n} is the inertia matrix, B∈Rⁿ is the vector of damping term, g∈Rⁿ is the gravitational torque and τ_{cmd}∈Rⁿ is the control torque command.

It is recognised that human motion is irregular, and fluctuations increase with duration when a low speed action/profile is desired, e.g. when a surgeon performs fine hand/finger movements during operation. With variable impedance control, the physical interaction can be improved as it regulates the impedance at high or low speed profiles. Therefore, the collaborative controller using variable admittance control, friction compensation gravity compensation for translational joints is proposed according to equations (7) to (9):

τ_{cmd}=τ_{ref}+τ_{fr}(V_{des})+τ_{gc} (7)

where τ_{ref} ∈Rⁿ is the reference torque input to be defined later with velocity and variable admittance controller. τ_{fr} (V_{des})∈Rⁿ is the desired friction compensation,

$$\{ \begin{matrix}{τ}_{f r} ( {V}_{d e s} ) = {τ}_{sta} , & if \vert {V}_{d e s} \vert < {V}_{th} \\ {τ}_{f r} ( {V}_{d e s} ) = {τ}_{C o u} , & others\end{matrix} \qquad ( 8 )$$

where V_{des}∈Rⁿ is the translation desired velocity, τₛₜₐ, τ_{Cou} are the statics and Coulomb friction, respectively, and Vₜₕ is the threshold velocity. To hold the platform of z-axis, a constant torque is applied as a gravity compensation.

τ_{gc}=[0 0 g_{cnt}]^{T} (9)

where g_{cnt} is the constant value for z-axis.

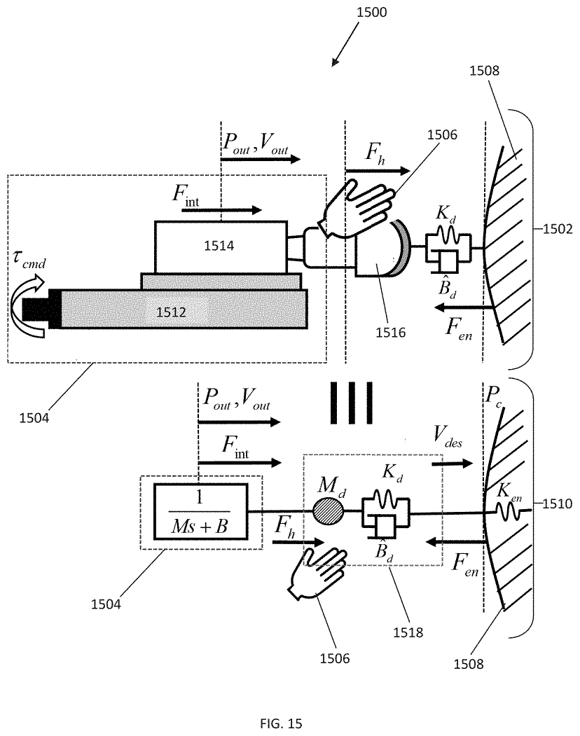

FIG. 15 is a schematic diagram 1500 illustrating modelling of a single axis (y-axis) with a control scheme in an exemplary embodiment. Top portion 1502 of the schematic diagram 1500 shows a robotic system 1504 operated by a user e.g. surgeon 1506 on a subject e.g. patient 1508 while bottom portion 1510 of the schematic diagram 1500 shows the modelling of the various components in the top portion 1502. The robotic system 1504 comprises a robot 1512 having a force/torque sensor 1514 and a probe e.g. ultrasound probe 1516. To simplify the discussion for τ_{ref}, only 1-DOF is considered in FIG. 15 as the 3 axes are decoupled. In the exemplary embodiment, delay time from the filters in sensor is taken into account in force signal processing loop and the US probe 1516 is mounted rigidly at the tip of the robot 1512. Hence, the robotic system 1504, F/T (force/torque) sensor 1514 and the US probe 1516 are considered as one single M, B system. The controller behaves as admittance 1518 (two force Fₕ and Fₑₙ in, desired velocity V_{des} out), with the desired mass, variable damping and spring (M_{d}, \hat{B}_{d}, K_{d}), regulating the interaction between the surgeon 1506, the robot 1512 and the patient 1508. The interaction force, Fᵢₙₜ, contributes to the desired force, F_{des}, by taking into account dead zone and saturation (see FIG. 12), triggering the robot motion which eventually results into velocity and position output, Vₒᵤₜ and Pₒᵤₜ, respectively.

The admittance with two interaction ports, Y(s), is described according to equation (10) as follows,

$$\begin{matrix}Y ( s ) = \frac{{V}_{des} ( s )}{{F}_{h} ( s ) - {F}_{e n} ( s )} \\ = \frac{{V}_{des} ( s )}{{F}_{des} ( s ) - {F}_{e n} ( s )} \\ = \frac{{V}_{des} ( s )}{Δ F ( s )} \\ ≡ \frac{s}{{M}_{d} {s}^{2} + {\hat{B}}_{d} s + {K}_{d}}\end{matrix} \qquad ( 10 )$$

Fₕ is surgeon's operation force, being obtained by the F/T sensor and filtered with signal processing into an interactive force, Fᵢₙₜ. The desired force, F_{des}, which is derived from Fᵢₙₜ, is applied for the collaborative controller. Fₑₙ is the environment reaction force from the patient. The force difference between two interaction ports is defined as ΔF(s).

Environment Force Estimation

Data that the F/T sensor retrieves are the net forces, including surgeon's operation force, the environmental reaction force and the interaction between the probe and sensor. As the probe is mounted rigidly with the F/T sensor, the interaction force in between can be treated as zero. Therefore, the remaining issue is to separate the environment force from the force sensor.

This may be achieved by mounting another force sensor measuring the environment force. However, this might not be feasible for the robot because the operational centre of the surgeon should intersect with the centre of the probe and robot to guarantee the decoupled motion design. To obtain the exact contacted environment force, the second sensor has to align with the rotational line of the end-effector where the first F/T sensor is. This distribution of two sensors increases the difficulty to separate the operational and environmental force apart. Besides, a multi-DOF force transmitter may not be cost-effective. Therefore, in the exemplary embodiment, the environment force is based on an estimation.

The first order model is assumed for the environment that exerts reaction force on the robot. The environment reaction force, Fₑₙ, is described according to equation (11) as follows, as shown in FIG. 15.

Fₑₙ=Kₑₙ(Pₒᵤₜ−P_{c}) (11)

Kₑₙ is the estimated stiffness of human skin or phantom, which is obtained experimentally. P_{c} is the central position of the contacted point.

Variable Admittance—Contacting and Tracking Axis (2-DOF)

The admittance, Y(s), from equation (10) is the control form for the two interaction ports. The desired mass, variable damping and spring, i.e., M_{d}, \hat{B}_{d}, and K_{d}, are the properties which regulate the interactive behaviours between these three objects, namely, the surgeon's hand, the robot with the probe and the patient. The goal of the variable admittance for the co-manipulation, is to vary the mass, damping and stiffness properties of the interaction ports in order to accommodate the human motion during the physical contacts with the robot and the patient. According to the experimental results, in general, when the operator performs relatively large movements at relatively high-speed profiles, low impedance parameters should be applied. High value of impedance, however, is more suitable for fine movements at low velocity. The desired (virtual) damping is vital for human's perception and the stability is mainly influenced by desired mass.

Therefore, assuming K_{d}=0 and fixed mass, M_{d}, only the desired damping, \hat{B}_{d}, is varied by these two interaction forces at the end-effector. Advantageously, the admittance designed in this way includes no zero in the transfer function, resulting in more stable performance. The admittance from equation (10) can be modified as,

$$\begin{matrix}Y ( s ) = \frac{1}{{M}_{d} s + {\hat{B}}_{d}} = \frac{{V}_{d e s} ( s )}{Δ F ( s )} \\ where & ( 12 ) \\ {\hat{B}}_{d} = {B}_{d} ( 1 \pm \vert Δ F ( s ) \vert \cdot α ) & ( 13 )\end{matrix}$$

where B_{d} is the constant damping within the stable range, a is the updated gain for this variable damping, \hat{B}_{d}, regulated by the force difference |ΔF| within two interaction ports.

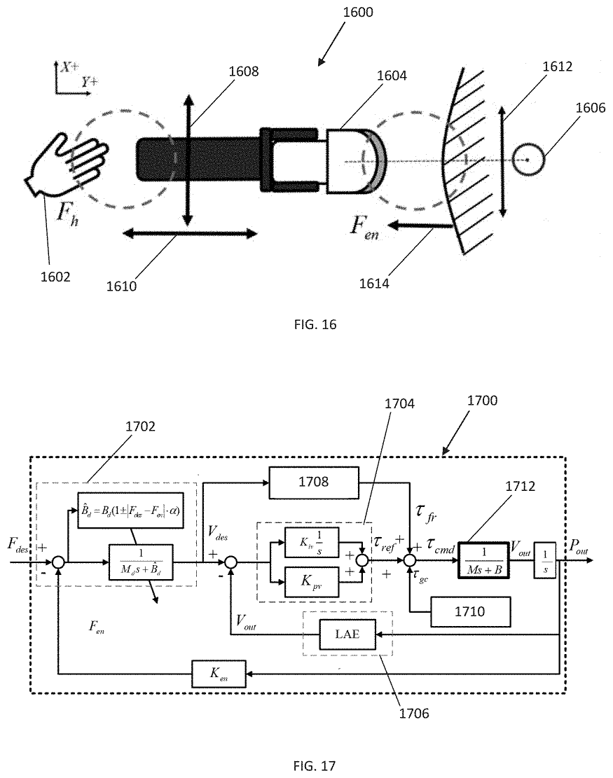

FIG. 16 is a schematic diagram 1600 illustrating two interaction port behaviours with 2 DOF axes in an exemplary embodiment. The schematic diagram 1600 shows a user e.g. surgeon 1602 operating an imaging probe e.g. ultrasound probe 1604 to scan a kidney stone or calyces 1606. As shown, a tracking axis is defined by 1608, a contacting axis is defined by 1610, and respiratory motion is defined by 1612. Bouncing of the probe 1604 from the surface is defined by arrow 1614. Conventionally, studies related to variable control claimed that low impedance is better for low velocity and high impedance should be applied at high speed (in the present case, low admittance for large force and high admittance for small force). This is true for the case that the operator guides the robot only with one interaction port (for example, line following and tracking) as human motion can be improved by high admittance for fine motion and vice versa. However, in the present embodiment, the updated equation to regulate \hat{B}_{d} should be different for the tracking and contacting axis for two interaction port behaviours, as shown in FIG. 16. In other words, the admittance in tracking (x) axis should decrease when the human force, Fₕ, is larger but contacting (y) axis should be opposite when the force difference, F, for two interaction ports changes. When it comes to two interaction ports with contacting environment, the desired dynamic behaviour to be achieved is regulating the force difference to generate a motion out. If the force difference between two interaction ports increases with high admittance, the controller exerts larger movement for the robot, resulting in two objects breaking the contacts. The main idea to design for a practical dynamic behaviour at the interaction port is where the robot exchanges energy with the objects or environment.

In summary of the above, the variable damping value from equation (13) is modified and applied as follows,

$$\{ \begin{matrix}{\hat{B}}_{d} = {B}_{d} ( 1 - \vert {F}_{des} ( s ) \vert \cdot α ) , & for x \& z axis \\ {\hat{B}}_{d} = {B}_{d} ( 1 + \vert Δ F ( s ) \vert \cdot α ) , & for y axis\end{matrix} \qquad ( 14 )$$

The new updated equations above correlate positively with the performance on the physical interaction behaviours between three objects in PCNL surgery. Namely, high admittance should be applied at large force difference in contacting axis, y-axis, and vice versa. The updated equation for admittance in tracking and maintaining axis, x-axis and z-axis, should remain the same with traditional studies to achieve higher accuracy with less execution time. The concepts for the two different updated equations in (14) above for variable admittance will be validated in the next section.

FIG. 17 is a schematic control block diagram of an admittance motion control loop 1700 for an individual translational joint in an exemplary embodiment, implementing the above updated equations. The admittance motion control loop 1700 comprises a variable admittance controller 1702, a velocity PI controller 1704, a velocity estimator 1706, a friction compensator 1708, a gravity compensator 1710, and a motor 1712 arranged according to the configuration of the control block diagram as shown in FIG. 17.

The control parameters are designed after the system identification. The characteristics of the designed controller are summarised in Table 1.

TABLE 1
Overview of the controller design

| | Trans- | | Updated | Gravity | |
|---|---|---|---|---|---|
| Joint | mission | Controller | equation | Compensation | Trigger |
| 1 | X | VA* | B_{d}(1 − \|F_{des}\| · α) | | FT |
| | | | | | sensor |
| 2 | Y | VA* | B_{d}(1 + ΔF · α) | | FT |
| | | | | | sensor |
| 3 | Z | VA* | B_{d}(1 − \|F_{des}\| · α) | V | FT |
| | | | | | sensor |
| 4 | R_{z} | Velocity | | | V_{des} = 0 |
| 5 | R_{y} | Velocity | | V | V_{des} = 0 |
| 6 | Rₓ | Torque | | | V_{des} = 0 |

*VA: Variable Admittance

### 2.2.1 Visual Servoing—Design Concept

In various exemplary embodiments, to address the limitations and gaps associated with PCNL surgery, an active control framework is proposed to track out-of-plane motion of the kidney stones during PCNL surgical procedure. It would be appreciated that even though the target application is PCNL surgery and involuntary movement of the patient is predominantly due to respiration, the proposed method can be generalized to different surgical tasks to compensate for involuntary movements which may be large enough to affect the outcomes of the surgical tasks.

Furthermore, the proposed method is capable of enhancing the ease of integration and operation because of two reasons. First, the proposed method can be readily implemented on any existing standard 2D ultrasound systems without any hardware modifications. Second, the active probe holding robotic manipulator takes care of maintaining correct contact force. This minimizes the need for human interaction and manual control of the ultrasound probe, allowing surgeon to focus on more critical tasks during the surgery. The proposed methodology for out-of-plane motion tracking comprises two major components namely, pre-scanning and Real-Time Visual Servoing (RTVS).

It would be appreciated that the pre-scan component may be replaced by pre-operative imaging of the target and constructing a model e.g. 3D model using the pre-operative images.

### 2.2.2 Visual Servoing—Out-of-Plane Motion Tracking

Pre-scan is the first step of an out-of-plane motion tracking framework that is used to construct missing 3D information around a target e.g. kidney stone. In this process, firstly, a user e.g. surgeon manually places the ultrasound probe tentatively at the centre of the target. A robotic manipulator which holds a 2D ultrasound probe then scans a small area around the target kidney stone. The purpose of performing a pre-scan is to record several consecutive B-mode ultrasound images at regular intervals to construct volume data with their position information.

Typically, PCNL surgery is done when the patient is in prone position as the lower pole calyces of the kidney are mostly, if not always subcostal. Therefore, proper selection of tracking axis for pre-scan is an important consideration especially in PCNL procedure. In order to create 3D volumetric data, there are four common scanning methods currently available—parallel, pivotal, tilt and rotational scanning. However, tilt and rotational scanning methods are presented with side/end firing transrectal (TRUS) probes which are commonly used in prostate imaging. A large region of interest can be scanned with a small angular displacement by tilting the conventional probe in a fan-like geometry using pivotal scanning method. However, the resolution of the acquired images tends to degrade with depth. This is an important consideration when it comes to selecting a suitable scanning modality for the proposed application as target kidney stone can be in anywhere inside a calyx. In contrast, parallel scanning method records a series of parallel 2D images by linearly translating the probe on patient's body without significantly affecting the image quality with depth. Hence, for pre-scanning using ultrasound, parallel scanning is used for pre-scan and subsequent real-time visual servoing.

Once the pre-scan is completed, the proposed system starts real-time tracking of out-of-plane motion of target kidneys stones. It has been recognised that there is a challenge in developing an out-of-plane motion tracking of kidney stones during PCNL surgery, as the calyceal anatomical structure around the target kidney stone can be symmetrical. Therefore, the images acquired from pre-scan to the left and right, while centre being the target are almost similar to each other. Although it is not an issue for one directional visual servoing, it poses a problem for two directional out-of-plane tracking. Therefore, a more practical approach is proposed herein to avoid the symmetrical problem by scanning the target area at an angle of 45° with respect to horizontal scan-line.

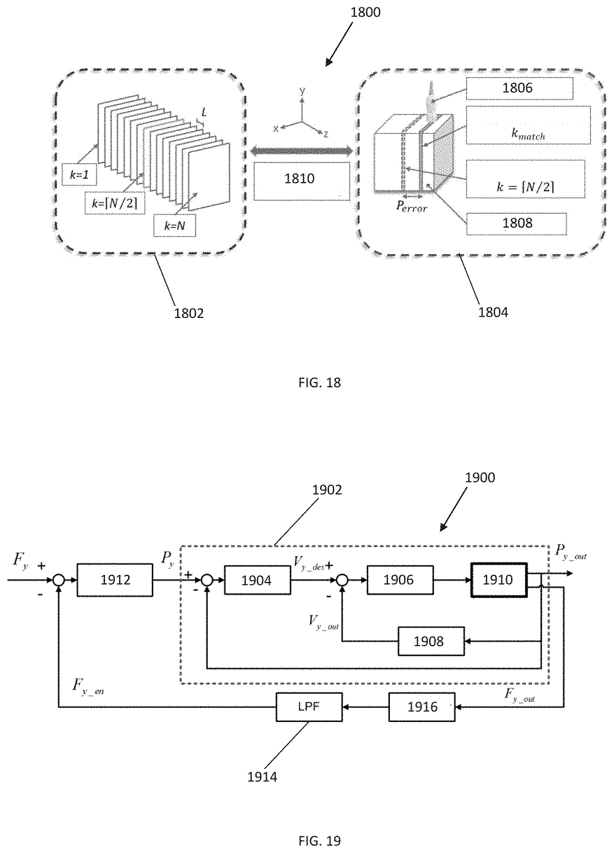

FIG. 18 is a schematic diagram showing an overview 1800 of out-of-plane motion tracking framework, including pre-scan 1802 and visual servoing 1804 stages in an exemplary embodiment. As a first step, a surgeon manually scans to locate the kidney area based on the information of preoperative images and places the ultrasound probe 1806 at the centre of the target (represented by centre frame index k=[N/2]). This is considered as the initial (home) position.

As a second step, a robotic manipulator (compare 904 of FIG. 9) moves the ultrasound probe 1806 by a distance of −L[N/2] from the initial position. Pre-scan data is being recorded while moving the probe 1806 by a distance of L(N−1) to scan a small region across the target kidney stone 1808. In the pre-scan, N consecutive frames at a regular interval of L are recorded to construct the 3D volume. After completing the pre-scan, robotic manipulator returns to its initial position.

As a third step, Real-Time Visual Servoing is performed. Inter-frame block matching 1810 is performed between the current frame (represented by current frame index k_{match}) and all N frames recorded from the pre-scan to find the best matched frame to the current frame. Sum of Squared Difference (SSD) is used as the similarity measure for the image correlation analysis. A rectangular region of interest (ROI) which includes the target kidney stone is selected for both current frame and pre-scanned frames to reduce the computational complexity of the block matching process. Calculation of SSD can be expressed as in equation (15)

SSD=Σⱼ₌₁ⁿΣᵢ₌₁^{m}{Iₖ(i,j)−I_{c}(i,j)}²k={0,1, . . . ,N} (15)

where Iₖ(i,j) and I_{c}(i,j) are the pixel intensity of the k^{th} frame and current frame respectively. m×n is the size of the rectangular ROI used. The best matched frame k is chosen by evaluating the index of the frame which has the lowest SSD(k) value. Hence, the position error of the current frame (P) (current location of the probe with respect to the initial position) along z-axis is estimated by

$${P}_{error} = ( {k}_{match} - ⌊ \frac{N}{2} ⌋ ) \times L \qquad ( 16 )$$

A predictive model is then applied to compensate the time delay between image processing and motion control loops. Then, the current position of the probe is estimated as

Z=Pₑᵣᵣₒᵣ+P_{delay} (17)

where P_{delay}=V (t_{delay}−T). V is defined as the velocity of the probe in the previous frame, t_{delay} and T are delay time in the TCP/IP loop and the sampling time respectively delay. Based on the estimated current position (Z), velocity command is given to the probe holding robot manipulator as in

V_{z}=γZ (18)

where γ is the gain of the vision controller. The objective of this method is to find the local minima of SSD values instead of calculating an exact value or a distance. Thus, inter-frame block matching is relatively robust for tracking out-of-plane motion of kidney stones compared to any conventional methods.

### 2.2.3 Visual Servoing—Position-Based Admittance Control Scheme

FIG. 19 is a schematic diagram of a proposed position-based admittance control scheme 1900 used to control a contact force between a probe and a body in an exemplary embodiment. The position-based admittance control scheme 1900 comprises a position control component 1902 which comprises a position controller 1904, a velocity controller 1906, a velocity estimator 1908, and a motor 1910 arranged as shown in FIG. 19. The position-based admittance control scheme 1900 further comprises an admittance controller 1912, a low pass filter (LPF) 1914, and a force/torque sensor 1916 connected to the position control component 1902 as shown in FIG. 19. The aim of admittance control is to control the dynamics of the contact surface to maintain the correct contact force with the patient's body. The control scheme 1900 for the environment contact is shown in FIG. 19, where F_{y} and F_{y_out} are the desired force and output force, respectively. F_{y_en} is the estimated environment force measured by the force/torque sensor with a 4^{th} order low pass filter (LPF), whose cut-off frequency is 2 Hz. P_{y}, V_{y}, P_{y_out} and V_{y_out} are the desired position, desired velocity, position output and velocity output, respectively.

The admittance controller, Y(s), can be described as in equation (19)

$$Y ( s ) = \frac{{P}_{y}}{dF} = \frac{{P}_{y}}{{F}_{y} - {F}_{y\_en}} ≡ \frac{1}{{B}_{d} s + {K}_{d}} \qquad ( 19 )$$

where dF is the force difference between the desired force and interactive force from the environment. B_{d} and K_{d} are the positive constants that represent desired damping and stiffness, respectively. Using a low pass filter, the environment force is delayed with a higher order transfer function. The target admittance is therefore designed as a first order system to prevent divergence due to inappropriate parameters. The admittance can be employed to achieve a desired force response with a low overshoot and small errors by tuning B_{d} and K_{d}. The robotic manipulator is designed with position control. Hence, the dynamic interaction between the robot and the environment can be regulated smoothly and the robot will move until the environment force is the same as the desired force.

It would be appreciated that pre-scan is a relatively robust method to gather missing 3D volume information of the surrounding area of the target e.g. kidney stone. However, this method is easily scalable so that the proposed Real-Time Visual Servoing (RTVS) algorithm can still be employed with minor modifications. This includes but is not limited to exploiting the periodic nature of the patient's respiration.

### 2.3 3D Anatomical Models Augmented Intra-Operative Guidance

In various exemplary embodiments, the apparatus for tracking a target in a body behind a surface may be used to perform 3D anatomical models augmented US-based intra-operative guidance. In other words, the apparatus may be used in conjunction with the method for registering real-time intra-operative data as described in FIG. 1 to FIG. 7. CT scanning may be performed in place of the pre-scan step, and is performed on the patient prior to the operation and boundaries of kidney, stones, and skin are semi-automatically delineated. All segmented models are then smoothed using a 3D Gaussian kernel and converted into triangulated meshes to generate approximated 3D anatomical models for downstream planning and guidance. Surgeons preoperatively plan and define a needle trajectory that avoids vital tissue and vessels to facilitate an effective treatment (which is suitable and safe for the interventional puncture). An optimal needle trajectory for the procedure can be defined as an entry point on the skin and a target point in the kidney.

During the surgical procedure, the ultrasound image slices of the kidney are acquired at the maximum exhalation positions of each respiratory circle to guide and visualise the needle position and orientation. The preoperatively generated 3D anatomical models and defined needle trajectory are then registered, using an affine 3D-2D registration algorithm, to the calibrated ultrasound images using a pair of orthogonal images. The kidney surface and cross-sectional shape of the kidney are used as registration features for the best alignment of the ultrasound image slices and the anatomical models. Since the transformation is calculated only at the maximum exhalation positions to counteract the effects of organ shift, soft-tissue deformation, and latency due to image processing on the registration, the accuracy of registered needle trajectory may not be guaranteed at the other stages of the respiratory circle. In view of the preceding, the puncture is performed at maximum exhalation positions. Generally, the needle entry on the skin is below the 12th rib, while avoiding all large vessels. By augmenting the clinically routine ultrasound images with the 3D preoperative anatomical models, preoperatively planned needle trajectory, and a virtual needle, a 3D visual intra-operative guidance is provided to facilitate an effective treatment (needle tracking in the case of robot-assisted surgery and the hand-eye coordination of the treating surgeon in the case of image-guided surgery).

### 2.4 Needle Insertion

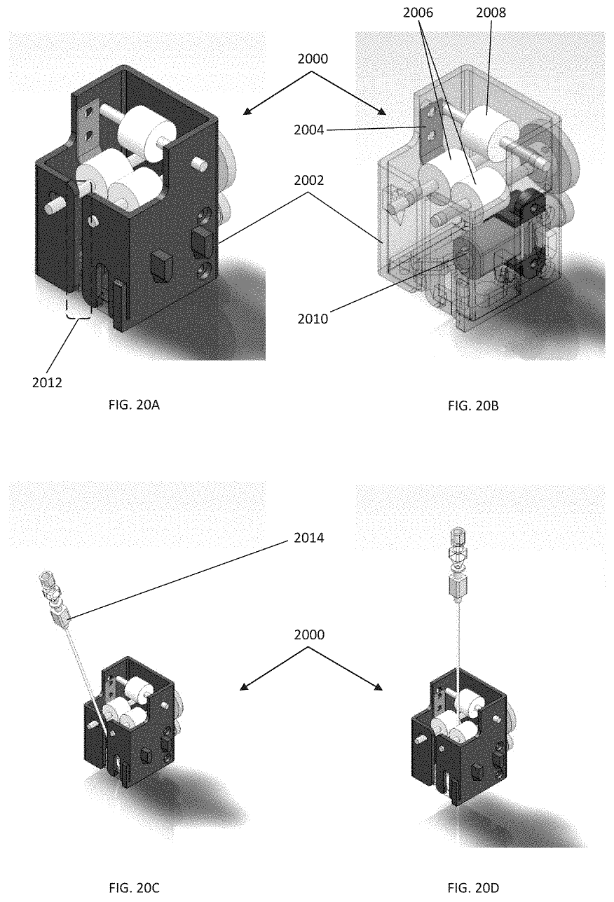

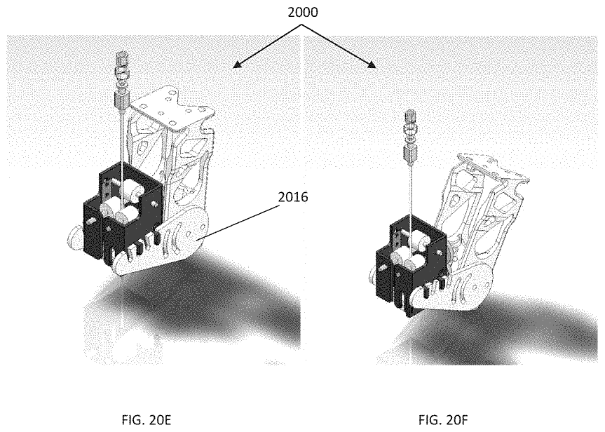

FIG. 20A is a perspective external view drawing of a needle insertion device (NID) 2000 in an exemplary embodiment. FIG. 20B is a perspective internal view drawing of the NID 2000 in the exemplary embodiment. FIG. 20C is a perspective view drawing of the NID 2000 having mounted thereon a needle in an angled orientation in the exemplary embodiment. FIG. 20D is a perspective view drawing of the NID 2000 having mounted thereon a needle in an upright orientation in the exemplary embodiment. FIG. 20E is a perspective view drawing of an assembly of the NID 2000 with an ultrasound probe mount at a first angle in the exemplary embodiment. FIG. 20F is a perspective view drawing of an assembly of the NID 2000 with the ultrasound probe mount at a second angle in the exemplary embodiment.

The NID 2000 comprises a casing 2002, a flat spring 2004 attached on the inner surface of the casing 2002, a pair of friction rollers 2006 and an additional friction roller 2008 arranged to receive and align a needle 2014, and a motor 2010 coupled to the friction rollers 2006 and 2008. A mounting slot 2012 is formed on the casing 2002 to allow side mounting/dismounting of the needle, as shown in FIG. 20C. Once the needle 2014 is mounted, the needle 2014 is oriented to its desired setup position as shown in FIG. 20D.

The NID 2000 utilises a friction drive transmission system, allows the needle to be controlled and manoeuvred automatically under the surveillance of the surgeon during percutaneous nephrolithotomy (PCNL) procedure. The friction rollers are driven by a Pololu micro DC motor (1:100 HP), with a rated output torque of 30 oz-in (0.21 N−m) at 6V. The motor can be removed from the bottom of the NID, allowing sterilization of the system. The flat spring 2004 is installed to ensure sure-contact of the needle to the pair of friction rollers 2006.

Movement of the friction rollers 2006 and 2008 can be controlled by an external microprocessor, including but not limited to rotation speed, duration of movement, and direction of motor rotation. A set of gears with a pre-determined gear ratio may be included to regulate the translational speed of the needle, therefore allowing precise movement of the needle. The mounting/side slot is designed to allow side mounting/dismounting of the needle, allowing the surgeon to perform subsequent manual operation without obstacle.

In the exemplary embodiment, a complementary imaging probe holder e.g. ultrasound probe holder 2016 may be included to form an assembly of the NID 2000 and an ultrasound probe, to ensure precise alignment of the NID 2000 to the ultrasound probe. Two different relative angles between the probe and the device can be selected based on surgeon's preference and/or procedure requirements, as shown in FIG. 20E and FIG. 20F.

In use, after out-of-plane motion of the kidney stones is compensated using the aforementioned methods, the in-plane motion of the needle tip is tracked to give a real-time visual feedback to the surgeon. This helps the surgeon to have a clear idea about the needle trajectory and complements for a successful initial needle puncture.

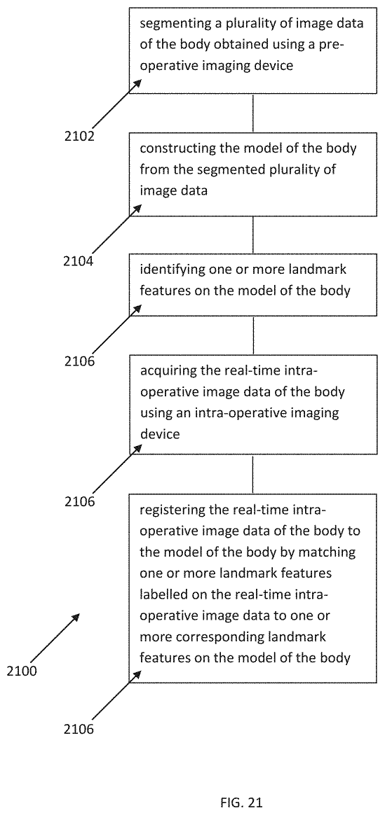

FIG. 21 is a schematic flowchart 2100 for illustrating a method for registering real-time intra-operative image data of a body to a model of the body in an exemplary embodiment. At step 2102, a plurality of image data of the body obtained using a pre-operative imaging device is segmented. At step 2104, the model of the body is constructed from the segmented plurality of image data. At step 2106, one or more landmark features are identified on the model of the body. At step 2108, the real-time intra-operative image data of the body is acquired using an intra-operative imaging device. At step 2110, the real-time intra-operative image data of the body is registered to the model of the body by matching one or more landmark features labelled on the real-time intra-operative image data to one or more corresponding landmark features on the model of the body. In the exemplary embodiment, the one or more landmark features comprises a superior and an inferior pole of the body.

In one exemplary embodiment, there is provided a robotic system for percutaneous nephrolithotomy to remove renal/kidney stones from a patient. The robotic system comprises an ultrasound probe for intra-operative 2D imaging, a stabilizing robotic manipulator which holds the ultrasound probe to maintain the correct contact force and minimise the need for human interaction and manual control of the ultrasound probe, and an automatic needle insertion device for driving a needle towards the target kidney stone. An admittance control algorithm is used to maintain an appropriate contact force between the ultrasound probe and the patient's body.

In the exemplary embodiment, the robotic system may be capable of performing ultrasound-guided visual servoing for involuntary motion compensation. To perform visual servoing, a semi-automated or user-guided segmentation of regions of interest is used to segment a series of pre-operative CT images of the kidney region. A 3-D model of the kidney and stone is then reconstructed from the segmented CT images for use in registering with real-time ultrasound images. Automated identification of anatomical landmarks or surface features is performed on the 3D reconstructed anatomical model of the kidney surface which can be localised and labelled in live ultrasound images. During percutaneous nephrolithotomy, the robotic system continuously updates and extracts a transformation matrix for transferring pre-operatively identified lesions to the live ultrasound images, so as to register the live ultrasound images and the 3D model. As an alternative to the 3D model from CT images, (high-resolution) scan images may be pre-obtained using real time ultrasound to construct a 3D volume of the kidney, which is then used for registration with intra-operative real-time ultrasound images.

In the exemplary embodiment, the automatic needle insertion device utilises a friction drive transmission system that allows the needle to be controlled and manoeuvred automatically under the surveillance of the surgeon during percutaneous nephrolithotomy.

In various exemplary embodiments as described herein, a method and system for registering real-time intra-operative image data of a body to a model of the body, as well as an apparatus for tracking a target in a body behind a surface using an intra-operative imaging device are used. The method and system may provide a semi-automated or user-guided segmentation of regions of interest e.g. kidney tissue from pre-operative images e.g. CT images. The method and system may further provide automated identification of anatomical landmarks or surface features on reconstructed anatomical model e.g. 3D model of the regions of interest e.g. kidney surface. The method and system may further provide a user-interface by which reliable anatomical landmarks can be localized and labelled in live intra-operative images e.g. ultrasound images. The method and system may further provide registration of the identified anatomical landmarks or surface features on the pre-operative anatomical model with the landmarks or features localized in the live intra-operative images e.g. ultrasound images. The method and system may further extract continuous updated transformation matrix for transferring pre-operatively identified features e.g. lesions to the live intra-operative images e.g. ultrasound images.

In use, the described exemplary embodiments of the system take the pre-operative images e.g. CT images as the input. Semi-automatic segmentation of the region of interest e.g. kidney tissue is performed after. The system is designed to allow segmentation and visualisation of multiple regions of interest (if any) to allow highlighting of lesions, if needed. Once done, the curvature-based feature extraction module kicks in to fit a tessellated surface, perform discrete curvature computation and localisation and labelling of pre-identified anatomical features (the same could be easily identified in 2D intra-operative images e.g. ultrasound images). Then, the system takes the real time intra-operative images e.g. 2D ultrasound images, pre-identified landmarks were seeded to allow the registration module to take over the process of registration. The system may be integrated to a computer aided surgical robot to guide a surgical or biopsy procedure intra-operatively based on a pre-planned procedure. The procedure can be removing an identified lesion or guide a tool to accurately biopsy a lesion for diagnostic purpose.

Described exemplary embodiments of the system are based on an intensity-based registration method which depends on similarity or higher-order image understanding. Advantageously, such intensity-based registration method may be better-suited for soft tissue structures such as bodily organs, as compared to a surface-based registration method which require ‘feature extraction’ of an artificial landmark inserted/placed physically into/near the body of interest for both imaging modalities (pre- and intra-operative). The resultant accuracy of surface-based registration methods is dependent on the robustness of the feature extraction, classification, and labelling algorithms, which makes it more suitable for robust surfaces like bones. The main difference and suitability between these two approaches is highly dependent on the anatomy, lesion, and procedure. In the described exemplary embodiments, the intensity-based registration method advantageously reduces the requirement of manual intervention during a procedure, considering no need for artificial/physical landmarks or markers, good accuracy through registration of surface instead of landmark points.

In the described exemplary embodiments, ultrasound imaging may be used for intra-operative imaging during procedures e.g. PCNL surgery. The use of intra-operative ultrasound may be feasible to achieve errors that satisfy the accuracy requirements of surgery. Ultrasound imaging may be accepted as a suitable imaging modality for diagnostic procedures due to its low cost and radiation free features. The equipment is also relatively small size, portable, and real time. Ultrasound imaging may be a convenient and safe alternative as an intra-operative imaging modality. In addition, ultrasound advantageously provides a real-time visualisation of not only the calyceal anatomy in 2 planes but also vital neighbouring organs, thus allowing a safe and accurate initial needle puncture. During PCNL, the surgeon is required to hold the ultrasound probe. Hand held ultrasound probe is preferred because it gives the surgeon the required flexibility and dexterity to have a clear access to the renal stone from various orientations and positions.

However, ultrasound image quality greatly suffers due to uncertainties of the scanning method—the probe must be kept directed at the target in a certain orientation for a considerable time until the surgeon makes a successful needle puncture to access the target calyx of the kidney. Another challenge that is imposed on the surgeon is that the surgeon has too many things to attend to during the procedure and each of these require full concentration. The surgeon has to hold the probe without creating unintended physiological movement while at the same handling other tasks. To complicate the situation, the kidney moves in its position due to the patient's respiration. In other words, the surgeon needs to hold the probe, look at the ultrasound images, decide the puncture location and insertion path, and perform the necessary insertion. US images also have some limitations in terms of low signal to noise ratio due to speckle, user-dependent acquisition and interpretation, and inability to penetrate bones. These spatial resolution limitations challenge the existing registration algorithms (3D surface models with live 2D images) and increase manual intervention steps during the process.

In the described exemplary embodiments, two important parameters for the success of the procedure using ultrasound imaging have been identified, namely: 1) maintaining the ultrasound probe position and orientation to correctly target a calyx or kidney stone despite the involuntary motion, and 2) achieving an appropriate contact force between the ultrasound probe and patient to obtain good quality ultrasound images.

In the described exemplary embodiments, the method for tracking a target in a body behind a surface using an intra-operative imaging device may be carried out using an apparatus/robot which has the following features: (1) a stabilizing manipulator, (2) ultrasound-guided visual servoing for involuntary motion compensation, (3) 3-D reconstruction of an anatomical model of the kidney and stone from CT images, and ultrasound-based intra-operative guidance, and (4) automatic needle insertion. The stabilizing manipulator may address the problem with unintended physiological movement while at the same allow the user to handling multiple tasks at the same time. The manipulator may be placed on a mobile platform that can be pushed near to the patient when required, so as anticipate potential issues of space constraint due to an additional manipulator in the surgical theatre. The ultrasound image-guided visual servoing method may provide tracking out-of-plane motion of the kidney stones influenced by the respiratory movement of the patient during PCNL surgery. In addition, an admittance control algorithm is proposed to maintain appropriate contact force between ultrasound probe and the patient's body when the operator releases the probe after initial manual positioning. This not only provides better image quality but also reduces burden on the surgeon so that he can concentrate on the more critical components.

The terms “coupled” or “connected” as used in this description are intended to cover both directly connected or connected through one or more intermediate means, unless otherwise stated.

The description herein may be, in certain portions, explicitly or implicitly described as algorithms and/or functional operations that operate on data within a computer memory or an electronic circuit. These algorithmic descriptions and/or functional operations are usually used by those skilled in the information/data processing arts for efficient description. An algorithm is generally relating to a self-consistent sequence of steps leading to a desired result. The algorithmic steps can include physical manipulations of physical quantities, such as electrical, magnetic or optical signals capable of being stored, transmitted, transferred, combined, compared, and otherwise manipulated.

Further, unless specifically stated otherwise, and would ordinarily be apparent from the following, a person skilled in the art will appreciate that throughout the present specification, discussions utilizing terms such as “scanning”, “calculating”, “determining”, “replacing”, “generating”, “initializing”, “outputting”, and the like, refer to action and processes of an instructing processor/computer system, or similar electronic circuit/device/component, that manipulates/processes and transforms data represented as physical quantities within the described system into other data similarly represented as physical quantities within the system or other information storage, transmission or display devices etc.

### 3. Computer System and Algorithm

The description also discloses relevant device/apparatus for performing the steps of the described methods. Such apparatus may be specifically constructed for the purposes of the methods, or may comprise a general purpose computer/processor or other device selectively activated or reconfigured by a computer program stored in a storage member. The algorithms and displays described herein are not inherently related to any particular computer or other apparatus. It is understood that general purpose devices/machines may be used in accordance with the teachings herein. Alternatively, the construction of a specialized device/apparatus to perform the method steps may be desired.

In addition, it is submitted that the description also implicitly covers a computer program, in that it would be clear that the steps of the methods described herein may be put into effect by computer code. It will be appreciated that a large variety of programming languages and coding can be used to implement the teachings of the description herein. Moreover, the computer program if applicable is not limited to any particular control flow and can use different control flows without departing from the scope of the invention.

Furthermore, one or more of the steps of the computer program if applicable may be performed in parallel and/or sequentially. Such a computer program if applicable may be stored on any computer readable medium. The computer readable medium may include storage devices such as magnetic or optical disks, memory chips, or other storage devices suitable for interfacing with a suitable reader/general purpose computer. In such instances, the computer readable storage medium is non-transitory. Such storage medium also covers all computer-readable media e.g. medium that stores data only for short periods of time and/or only in the presence of power, such as register memory, processor cache and Random Access Memory (RAM) and the like. The computer readable medium may even include a wired medium such as exemplified in the Internet system, or wireless medium such as exemplified in bluetooth technology. The computer program when loaded and executed on a suitable reader effectively results in an apparatus that can implement the steps of the described methods.

The exemplary embodiments may also be implemented as hardware modules. A module is a functional hardware unit designed for use with other components or modules. For example, a module may be implemented using digital or discrete electronic components, or it can form a portion of an entire electronic circuit such as an Application Specific Integrated Circuit (ASIC). A person skilled in the art will understand that the exemplary embodiments can also be implemented as a combination of hardware and software modules.

Additionally, when describing some embodiments, the disclosure may have disclosed a method and/or process as a particular sequence of steps. However, unless otherwise required, it will be appreciated the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For an example, when “comprising” is used, reference to a “one” feature is also intended to be a reference to “at least one” of that feature. Terms such as “consisting”, “consist”, and the like, may, in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

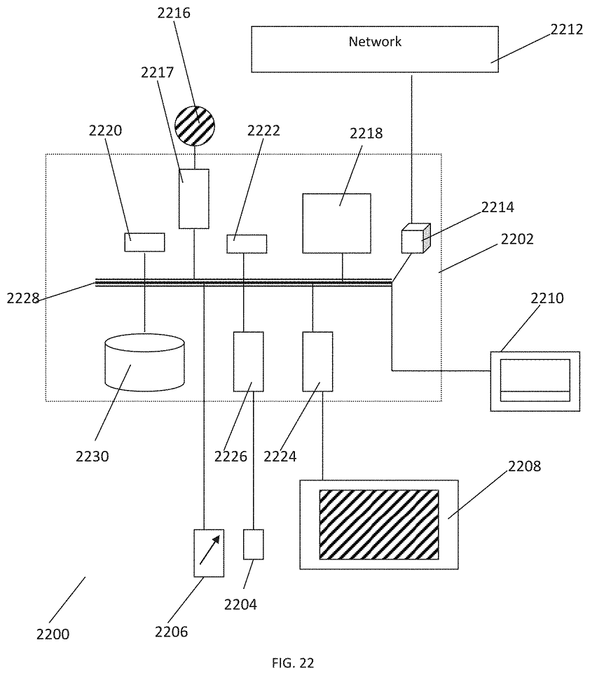

Different exemplary embodiments can be implemented in the context of data structure, program modules, program and computer instructions executed in a computer implemented environment. A general purpose computing environment is briefly disclosed herein. One or more exemplary embodiments may be embodied in one or more computer systems, such as is schematically illustrated in FIG. 22.

One or more exemplary embodiments may be implemented as software, such as a computer program being executed within a computer system 2200, and instructing the computer system 2200 to conduct a method of an exemplary embodiment.

The computer system 2200 comprises a computer unit 2202, input modules such as a keyboard 2204 and a pointing device 2206 and a plurality of output devices such as a display 2208, and printer 2210. A user can interact with the computer unit 2202 using the above devices. The pointing device can be implemented with a mouse, track ball, pen device or any similar device. One or more other input devices (not shown) such as a joystick, game pad, satellite dish, scanner, touch sensitive screen or the like can also be connected to the computer unit 2202. The display 2208 may include a cathode ray tube (CRT), liquid crystal display (LCD), field emission display (FED), plasma display or any other device that produces an image that is viewable by the user.

The computer unit 2202 can be connected to a computer network 2212 via a suitable transceiver device 2214, to enable access to e.g. the Internet or other network systems such as Local Area Network (LAN) or Wide Area Network (WAN) or a personal network. The network 2212 can comprise a server, a router, a network personal computer, a peer device or other common network node, a wireless telephone or wireless personal digital assistant. Networking environments may be found in offices, enterprise-wide computer networks and home computer systems etc. The transceiver device 2214 can be a modem/router unit located within or external to the computer unit 2202, and may be any type of modem/router such as a cable modem or a satellite modem.

It will be appreciated that network connections shown are exemplary and other ways of establishing a communications link between computers can be used. The existence of any of various protocols, such as TCP/IP, Frame Relay, Ethernet, FTP, HTTP and the like, is presumed, and the computer unit 2202 can be operated in a client-server configuration to permit a user to retrieve web pages from a web-based server. Furthermore, any of various web browsers can be used to display and manipulate data on web pages.

The computer unit 2202 in the example comprises a processor 2218, a Random Access Memory (RAM) 2220 and a Read Only Memory (ROM) 2222. The ROM 2222 can be a system memory storing basic input/output system (BIOS) information. The RAM 2220 can store one or more program modules such as operating systems, application programs and program data.

The computer unit 2202 further comprises a number of Input/Output (I/O) interface units, for example I/O interface unit 2224 to the display 2208, and I/O interface unit 2226 to the keyboard 2204. The components of the computer unit 2202 typically communicate and interface/couple connectedly via an interconnected system bus 2228 and in a manner known to the person skilled in the relevant art. The bus 2228 can be any of several types of bus structures including a memory bus or memory controller, a peripheral bus, and a local bus using any of a variety of bus architectures.

It will be appreciated that other devices can also be connected to the system bus 2228. For example, a universal serial bus (USB) interface can be used for coupling a video or digital camera to the system bus 2228. An IEEE 1394 interface may be used to couple additional devices to the computer unit 2202. Other manufacturer interfaces are also possible such as FireWire developed by Apple Computer and i.Link developed by Sony. Coupling of devices to the system bus 2228 can also be via a parallel port, a game port, a PCI board or any other interface used to couple an input device to a computer. It will also be appreciated that, while the components are not shown in the figure, sound/audio can be recorded and reproduced with a microphone and a speaker. A sound card may be used to couple a microphone and a speaker to the system bus 2228. It will be appreciated that several peripheral devices can be coupled to the system bus 2228 via alternative interfaces simultaneously.

An application program can be supplied to the user of the computer system 2200 being encoded/stored on a data storage medium such as a CD-ROM or flash memory carrier. The application program can be read using a corresponding data storage medium drive of a data storage device 2230. The data storage medium is not limited to being portable and can include instances of being embedded in the computer unit 2202. The data storage device 2230 can comprise a hard disk interface unit and/or a removable memory interface unit (both not shown in detail) respectively coupling a hard disk drive and/or a removable memory drive to the system bus 2228. This can enable reading/writing of data. Examples of removable memory drives include magnetic disk drives and optical disk drives. The drives and their associated computer-readable media, such as a floppy disk provide nonvolatile storage of computer readable instructions, data structures, program modules and other data for the computer unit 2202. It will be appreciated that the computer unit 2202 may include several of such drives. Furthermore, the computer unit 2202 may include drives for interfacing with other types of computer readable media.

The application program is read and controlled in its execution by the processor 2218. Intermediate storage of program data may be accomplished using RAM 2220. The method(s) of the exemplary embodiments can be implemented as computer readable instructions, computer executable components, or software modules. One or more software modules may alternatively be used. These can include an executable program, a data link library, a configuration file, a database, a graphical image, a binary data file, a text data file, an object file, a source code file, or the like. When one or more computer processors execute one or more of the software modules, the software modules interact to cause one or more computer systems to perform according to the teachings herein.

The operation of the computer unit 2202 can be controlled by a variety of different program modules. Examples of program modules are routines, programs, objects, components, data structures, libraries, etc. that perform particular tasks or implement particular abstract data types. The exemplary embodiments may also be practiced with other computer system configurations, including handheld devices, multiprocessor systems, microprocessor-based or programmable consumer electronics, network PCs, minicomputers, mainframe computers, personal digital assistants, mobile telephones and the like. Furthermore, the exemplary embodiments may also be practiced in distributed computing environments where tasks are performed by remote processing devices that are linked through a wireless or wired communications network. In a distributed computing environment, program modules may be located in both local and remote memory storage devices.

The exemplary embodiments may also be practiced with other computer system configurations, including handheld devices, multiprocessor systems/servers, microprocessor-based or programmable consumer electronics, network PCs, minicomputers, mainframe computers, personal digital assistants, mobile telephones and the like. Furthermore, the exemplary embodiments may also be practiced in distributed computing environments where tasks are performed by remote processing devices that are linked through a wireless or wired communications network. In a distributed computing environment, program modules may be located in both local and remote memory storage devices.

It will be appreciated by a person skilled in the art that other variations and/or modifications may be made to the specific embodiments without departing from the scope of the invention as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1-51. (canceled)

52. A method for registering real-time intra-operative image data of a body to a model of the body, the method comprising,
segmenting a plurality of image data of the body obtained using a pre-operative imaging device;
constructing the model of the body from the segmented plurality of image data;
identifying one or more landmark features on the model of the body;
acquiring the real-time intra-operative image data of the body using an intra-operative imaging device; and
registering the real-time intra-operative image data of the body to the model of the body by matching one or more landmark features labelled on the real-time intra-operative image data to one or more corresponding landmark features on the model of the body,
wherein the one or more landmark features comprises a superior and an inferior pole of the body, and a line connecting the superior and inferior poles of the body.

53. The method of claim 52, wherein the one or more landmark features further comprises a combination of saddle ridge, saddle valley, peak and/or pit.

54. The method of claim 52, wherein the step of identifying one or more landmark features comprises calculating one or more principal curvatures for each vertex of the body.

55. The method of claim 54, wherein the step of identifying one or more landmark features further comprises calculating the Gaussian and mean curvatures using the one or more principal curvatures, wherein the one or more landmark features is identified by a change in sign of the Gaussian and mean curvatures.

56. The method of claim 52, further comprising labelling one or more landmark features on the real-time intra-operative image data using a user interface input module.

57. The method of claim 52, further comprising sub-sampling or down-sampling of the model to match the resolution of the real-time intra-operative image data acquired by the intra-operative imaging device.

58. The method of claim 52, wherein the step of registering comprises iteratively reducing the Euclidean distance between the one or more landmark features labelled on the real-time intra-operative image data of the body and the one or more corresponding landmark features on the model of the body.

59. The method of claim 52, wherein the step of registering comprises matching the superior and inferior poles of the body on the real-time intra-operative image data to the respective superior and inferior poles of the body on the model of the body.

60. The method of claim 52, wherein the step of segmenting comprises introducing one or more seed points in one or more regions of interest, wherein each of the one or more seed points comprises a pre-defined threshold range of pixel intensities.

61. The method of claim 60, further comprising iteratively adding to the one or more seed points, neighbouring voxels with pixel intensities within the pre-defined threshold range of pixel intensities of the one or more seed points.

62. The method of claim 52, further comprising generating a polygonal mesh of the model to render the model for visualization on a display screen, wherein the polygonal mesh is a triangular or quadrilateral mesh.

63. The method of claim 52, wherein the pre-operative imaging device is a computed tomography (CT) imaging device, a magnetic resonance (MR) imaging device, or an ultrasound imaging device.

64. The method of claim 52, wherein the intra-operative imaging device is an ultrasound imaging device.

65. The method of claim 52, wherein the body is located within a human or an animal.

66. The method of claim 65, further comprising labelling the one or more landmark features on the real-time intra-operative image data at substantially the same point in a respiratory cycle of the human or animal body.

67. The method of claim 66, wherein the point in the respiratory cycle of the human or animal body is the point of substantially maximum exhalation.

68. The method of claim 52, wherein the body is a kidney.

69. A system for registering real-time intra-operative image data of a body to a model of the body, the system comprising,
an image processing module configured to:
segment a plurality of image data of the body obtained using a pre-operative imaging device;
construct the model of the body from the segmented plurality of image data;
identify one or more landmark features on the model of the body;
an intra-operative imaging device configured to acquire the real-time intra-operative image data of the body; and
a registration module configured to register the real-time intra-operative image data of the body to the model of the body by matching one or more landmark features labelled on the real-time intra-operative image data to one or more corresponding landmark features on the model of the body,
wherein the one or more landmark features comprises a superior and an inferior pole of the body, and a line connecting the superior and inferior poles of the body.

70. The system of claim 69, further comprising a pre-operative imaging device for acquiring a plurality of image data of the body,
wherein the pre-operative imaging device is a computed tomography (CT) imaging device, a magnetic resonance (MR) imaging device, or an ultrasound imaging device; and
wherein the intra-operative imaging device is an ultrasound imaging device.

71. An apparatus for tracking a target in a body behind a surface using an intra-operative imaging device, the intra-operative imaging device comprising a probe for performing scans of the body, and an image feedback unit for providing real-time intra-operative image data of the scans obtained by the probe, the apparatus comprising,
a manipulator for engaging and manipulating the probe;
a control unit for positioning the probe by controlling the manipulator, said control unit comprising,
an image processing module configured to:
segment a plurality of image data of the body obtained using a pre-operative imaging device;
construct a model of the body from the segmented plurality of image data, said model comprising an optimal needle trajectory information, and said optimal needle trajectory information comprising positional information on a point on the surface and a point of the target;
identify one or more landmark features on the model of the body;
a registration module configured to register the real-time intra-operative image data of the body to the model of the body by matching one or more landmark features labelled on the real-time intra-operative image data to one or more corresponding landmark features on the model of the body, wherein the one or more landmark features comprises a superior and an inferior pole of the body, and a line connecting the superior and inferior poles of the body; and
a needle insert device coupled to the manipulator, said needle insert device comprising holding means for holding a needle at an angle directed at the target;
wherein said manipulator is configured to directly manipulate the probe in collaboration with the control unit such that the needle substantially follows the optimal needle trajectory information to access the target in the body; and
wherein the control unit is configured to address undesired motion of the probe caused by the user or the body of the target.
